(19) United States
(12) Patent Application Publication
(10) Pub. No.: US 2005/0147977 A1
(43) Pub. Date: Jul. 7, 2005

(54) Methods and compositions for nucleic acid detection and sequence analysis

(76) Inventors: Tae-Woong Koo, South San Francisco, CA (US); Selena Chan, San Jose, CA (US)

Correspondence Address: DLA PIPER RUDNICK GRAY CARY US, LLP, 4365 EXECUTIVE DRIVE, SUITE 1100, SAN DIEGO, CA, 92121-2133, US

(21) Appl. No.: 10/748,525

(22) Filed: Dec. 29, 2003

(51) Int. Cl. C12Q 1/68; C07H 21/04

(52) U.S. Cl. 435/6; 536/24.3

(57) ABSTRACT

A population of labeled probes is provided that utilize an encoding system in which both the intensity and specific characteristics of a signal molecule are utilized to reduce the number of signal molecules necessary to identify each member of the population of probes. In the population of labeled probes, each labeled probe includes a probe associated with a series of detectably distinguishable signal molecules. The number and type of signal molecules identifies the associated probe, and the number of probes in the population exceeds the number of unique signal molecules. The population of probes are used in methods of the invention and reaction mixtures of the invention, for identifying a target molecule and for sequencing a nucleic acid molecule, for example.

### BACKGROUND OF THE INVENTION

1. Field of the Invention

The invention relates generally to data encoding and more specifically to encoding biomolecular information.

2. Background Information

The medical field, among others, is increasingly in need of techniques for identification and characterization of biomolecules. In particular, techniques for detecting and/or sequencing multiple DNA molecules in a single reaction have become more important due in part to recent medical advances utilizing genetics and gene therapy.

The ability to detect multiple biomolecules in a single reaction or detect a single biomolecule using multiple probes becomes more important as additional genes, proteins, and variants are identified. Multiplex analysis typically involves utilization of multiple probes in a single reaction. Currently, gene probes for optical detection utilize one type of signal molecule. Thus, present multiplex technologies are limited by the limited number of signal molecules available.

The significance of this limitation becomes even more apparent with respect to nucleic acid sequence analysis. When it is desired to test whether a target nucleic acid strand contains a specific sequence of nucleotides, oligonucleotide probes can be used. Hybridization and detection of an oligonucleotide probe to a target nucleic acid strand indicates that the target nucleic acid strand contains a nucleic acid sequence complementary to the hybridized oligonucleotide probe. If the oligonucleotide probe has n-nucleotides, referred to as an n-mer, there are 4ⁿ possible nucleic acid sequences. If one type of signal molecule is used to represent one nucleic acid sequence, as is the case with present methods (See e.g., Vo-Dinh et al, J. Raman Spectrosc., 30: 785-793 (1999); Graham et al, Anal. Chem., 74:1069-1074 (2002), Mirkin et al, Science, 297: 1536-1 540 (2002)), 4ⁿ types of signal molecules are necessary. Accordingly, 4²⁰ (˜10\hat{ }12) types of signal molecules are necessary to represent all possible variations of a 20-mer (n=20). Thus, as has been suggested, more than a trillion types of signal molecules must be used in traditional methods, to produce a matching number of gene probes for multiplex analysis (See e.g., Vo-Dinh et al, 1999). However, such methods suffer from a limited number of available label molecules and difficulties in detecting large numbers of label molecules in a single reaction.

In addition to problems created by the number of signal molecules necessary for multiplex assays, when multiple signal molecules are used, additional problems arise. For example, it is difficult to determine the order of individual signal molecules when they are bound to a probe. For example, a 20-mer is approximately 7 nm long, which is smaller than a typical diffraction limit of a far field optical instruments (˜400 nm), or a typical resolution of near-field optical instruments (50-200 nm). Thus, it is difficult to code information regarding a probe using the order of a limited number of signal molecules bound to the probe.

Furthermore, when using scanning probe microscopy to detect nanotags, the tags can have different geometric configurations due to bending, torsion, and stretching. Therefore, it is difficult to identify the order of nanotags, and thus, difficult to code information regarding a probe based on an order of nanotags on the probe. Accordingly, a need exists for methods of encoding data to reduce the number of signal molecules that do not depend upon the order of nanotags.

### BRIEF DESCRIPTION OF THE DRAWINGS

FIGS. 1A-1D illustrate a theoretical spectra of a reference molecule and signal molecules, when each signal molecule has a unique peak. FIG. 1A shows a theoretical spectrum of a theoretical reference molecule. FIG. 1B shows a theoretical spectrum of a first encoding signal molecule. FIG. 1C shows a theoretical spectrum of a second encoding signal molecule. FIG. 1D shows a theoretical spectrum of a third encoding signal molecule.

FIGS. 2A-2D illustrate exemplary hypothetical spectra of tags. Based on the peak positions and intensity, the number of encoding signal molecules can be calculated. FIG. 2A shows a 1:1:1 ratio of 3 encoding signal molecules compared to a reference molecule. FIG. 2B shows a 1:2:0 ratio of 3 encoding signal molecules compared to a reference molecule. FIG. 2C shows a 4:1:2 ratio of 3 encoding signal molecules compared to a reference molecule. FIG. 2D shows a 3:3:3 ratio of 3 encoding signal molecules compared to a reference molecule.

### DETAILED DESCRIPTION OF THE INVENTION

The present invention is based on the discovery of an encoding approach that reduces the of signal molecules that are required to encode information about a probe and its target. Thus, the present invention allows more probes to be distinguished using fewer types of signal molecules. The approach uses both the intensity and specific identity of a signal generated from signal molecules to identify one or more labeled probes associated with the signal molecules. This allows labeling of probes with fewer signal molecules than if each probe was labeled with a unique signaling molecule. Furthermore, it allows for encoding a large number of probes using signal molecules, without the need to determine the order of signal molecules on the probe.

Accordingly, a method is provided for identifying a nucleotide sequence of a target nucleic acid by contacting the target nucleic acid with a population of labeled oligonucleotide probes, wherein each labeled oligonucleotide probe includes a series of detectably distinguishable signal molecules associated with an oligonucleotide, wherein the oligonucleotide is identifiable by the number and type of associated signal molecules, and wherein the number of probes exceeds the number of unique signal molecules. The bound oligonucleotide probes are separated from unbound labeled oligonucleotide probes. A signal generated from the bound labeled oligonucleotide probes is detected and decomposed to identify the number and type of signal molecules in the bound labeled oligonucleotide probes, thereby identifying a nucleotide sequence of the target nucleic acid.

As discussed in further detail herein, the labeled oligonucleotide probes include one or more labels that are typically covalently attached to each oligonucleotide. The oligonucleotide can be labeled at one nucleotide, or it can be labeled at more than one nucleotide. Furthermore, one or more labels can be attached to each nucleotide that is labeled.

In certain aspects, each unique signal molecule is present up to 4 times per labeled oligonucleotide probe. In these aspects, for example, the number of unique signal molecules is equal to the number of nucleotides of the labeled oligonucleotide probe. Furthermore, the nucleotide occurrence of each nucleotide position of the labeled oligonucleotide probe can be identified by a number of copies of each signal molecule, for example.

In certain aspects of the invention, each labeled oligonucleotide probe includes an intensity reference signal molecule. As discussed in further detail herein, the intensity reference signal molecule can assist in a determination of the detected number of copies of a signal molecule. The signal molecules can be Raman labels, fluorescent labels, quantum dots, or nanoparticles, for example, as discussed in more detail herein. Intensity reference signal molecules also help to differentiate signals generated from multiple copies of a label from signals generated from labels that include multiple copies of other labels (see e.g., the label encoding AAA and GGG in Table 1).

In certain aspects, the population of labeled oligonucleotide probes includes all possible sequence combinations of an oligonucleotide of the identical length. These aspects are used, for example, with sequencing by hybridization methods. A sequencing by hybridization method using the population of labeled oligonucleotide probes disclosed herein, for example, can include a second population of probes, a population of capture probes. As discussed in more detail herein, capture probes are nucleic acid molecules with known nucleotide sequences. These probes are synthesized by standard chemical methods and can be optionally labeled. Capture probes are typically immobilized on a solid surface at either their 5′ or 3′ end. Standard chemical cross linking techniques can be used for probe immobilization, such as thiol-gold linkage or amine-aldehyde linkage. Methods for immobilization of nucleic acids are disclosed in more detail herein.

Accordingly, in sequencing by hybridization aspects provided herein, a method for determining a nucleotide sequence of a target nucleic acid includes contacting the nucleic acid, or a fragment thereof, with a population of capture oligonucleotide probes bound to a substrate at a series of spot locations, to form a probe-target duplex polynucleotides comprising single-stranded overhangs, contacting the probe-target duplex nucleic acids with a population of labeled oligonucleotide probes as disclosed herein, to allow binding of the labeled oligonucleotide probes to the single-stranded overhangs, and detecting labeled oligonucleotide probes that bind the target nucleic acid, thereby determining a nucleotide sequence of the target nucleic acid. Furthermore, the location of the spot for each of the captured labeled oligonucleotide probes can be identified and used to determine the nucleotide sequence of the target nucleic acid.

In certain aspects directed at sequencing by hybridization, the method further includes an optional ligation reaction. The ligation reaction typically involves ligation of a capture oligonucleotide probe to a labeled oligonucleotide probe that binds to adjacent regions of a target nucleic acid. After adjacent oligonucleotides are ligated, oligonucleotides that are not immobilized to the substrate can be removed, for example by elevating the temperature or changing the pH of a reaction to denature nucleic acids. Oligonucleotides that are not immobilized to the substrate either directly or indirectly can be washed away and the immobilized oligonucleotides can be detected. The ligation and wash steps increase the specificity of the reaction.

Accordingly, capture oligonucleotide probes can be immobilized on various spots on a substrate. In aspects that include a ligation step, a labeled oligonucleotide probe ligates to a capture oligonucleotide probe only when the target nucleic acid includes target segments that are complementary to both the Raman-active oligonucleotide probe and the capture oligonucleotide probe, respectively, and the two segments are adjacent to each other. In this aspect, the nucleotide sequence is determined based on a detected signal from the ligated labeled oligonucleotide probes and the corresponding positions of capture probes.

Adjacent labeled oligonucleotide probes can be ligated together using known methods (see, e.g., U.S. Pat. Nos. 6,013,456). Primer independent ligation can be accomplished using oligonucleotides of at least 6 to 8 bases in length (Kaczorowski and Szybalski, Gene 179:189-193, 1996; Kotler et al., Proc. Natl. Acad. Sci. USA 90:4241-45, 1993). Methods of ligating oligonucleotide probes that are hybridized to a nucleic acid template are known in the art (U.S. Pat. No. 6,013,456). Enzymatic ligation of adjacent oligonucleotide probes can utilize a DNA ligase, such as T4, T7 or Taq ligase or E. coli DNA ligase. Methods of enzymatic ligation are known (e.g., Sambrook et al., 1989).

The population of labeled oligonucleotide probes can be modified such that they cannot be ligated at their 3′ end to another labeled oligonucleotide probe. This helps to eliminate ambiguity of differentiating labels that include multiple copies of other labels (see e.g., the label encoding AAA and GGG in Table 1), since it assures that a signal generated from labeled oligonucleotide probes at a capture probe spot, is generated only from individual labeled oligonucleotide probes. For example, labeled oligonucleotide probes can be modified to include a dideoxy nucleotide at the 3′ end to block ligation of labeled oligonucleotide probes.

In another embodiment, the present invention provides a population of labeled probes that include a probe associated with a series of detectably distinguishable signal molecules, also referred to herein as labels, wherein the number and type of signal molecules identifying the associated probe, and wherein the number of probes in the population exceeds the number of unique signal molecules. This property of the population of labeled probes provides an advantage over known methods because fewer signal molecules are required than traditional methods, which require one signal molecule for every probe in a population of probes.

The probe molecule is a specific binding pair member, for example, a nucleic acid, such as an oligonucleotide or a polynucleotide; a protein or peptide fragment thereof, such as a receptor or a transcription factor, an antibody or an antibody fragment, for example, a genetically engineered antibody, a single chain antibody, or a humanized antibody; a lectin; a substrate; an inhibitor; an activator; a ligand; a hormone; a cytokine; a chemokine; and/or a pharmaceutical. The probe molecules can be used to detect a variety of target molecules such as polynucleotides and polypeptides, and combinations thereof, as discussed in more detail herein.

In certain aspects, the probe molecule is an oligonucleotide, wherein the nucleotide sequence is identified by the number and type of signal molecules associated with the oligonucleotide probe. The population of labeled oligonucleotide probes are also referred to herein as a “labeled oligonucleotide library.” The population of oligonucleotides are typically hybridization probes that include a known nucleotide sequence portion, also referred to as a probe portion, associated with a series of detectably distinguishable signal molecules. The oligonucleotides are useful, for example, for sequencing by hybridization reactions, or for other types of hybridization reactions.

In certain aspects the population includes oligonucleotides with nucleotide sequences that correspond to every possible permutation less than or equal to the length of the oligonucleotides. The length of the oligonucleotide portion can be varied based on the particular requirements for detection. However, in certain aspects all of the nucleotides in the population are of an identical length. For example, the labeled oligonucleotide can be equal to or less than 250 nucleotides, 200 nucleotides, 100 nucleotides, 50 nucleotides, 25 nucleotides, 20 nucleotides, 15 nucleotides, 10 nucleotides, 9 nucleotides, 8 nucleotides, 7 nucleotides, 6 nucleotides, 5 nucleotides, 4 nucleotides, or 3 nucleotides in length. For example, but not intended to be limiting, the oligonucleotide is 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 125, 150, 200, or 250 nucleotides in length. For example, the population of oligonucleotide probes can be an identical length of between about 3 and 25 nucleotides in length. In other aspects, the population of oligonucleotide probes are an identical length of between about 10 and about 50 nucleotides.

The population of labeled oligonucleotides in certain aspects, includes at least 10, 20, 30, 40, 50, 100, 200, 250, 500, 1000, oligonucleotides. For example, the population can include substantially all, or all of the possible nucleotide sequence combination for oligonucleotides of an identical length, as is known for at least some sequencing by hybridization reactions (See e.g., U.S. Pat. No. 5,002,867). Substantially all of the possible nucleotide sequence combinations for a given length include enough of the possible nucleotide sequences to allow unequivocal detection of a hybridizing target nucleic acid.

The series of detectably distinguishable signal molecules are, for example, a series of signal molecules that are detectable by optical methods, detectable by scanning probe methods, and/or detectable using an electron microscope. The signal molecules are distinguishable from each other such that the specific number and identity of each signal molecule can be determined even when detecting a population probes that includes all of the signal molecules. In certain aspects, the labeled probes include one or more linkers that link two signal molecules and/or the probe and the signal molecule, as discussed in more detail herein.

The labeled probes of the present invention can be detected for example, by single molecule level detection methods or by scanning probe microscopy methods, both of which can be non-optical or optical methods. For example, for optical detection the signal molecules can be a series of dye molecules that can be detected using fluorescence or surface enhanced Raman spectroscopy (SERS), or both. In certain aspects, the series of signal molecules, for example, are Raman active polymethine dyes (K. Kneipp et al. Chem. Reviews (1999). Polymethine dye molecules can be selected which have unique Raman spectra and which can be relatively easily differentiated.

In aspects of the present invention where the labeled probes are detected using optical detection, intensity information is used, in addition to the specific detected optical signal. The intensity information provides additional information in order to increase the number of probes that can be represented by a combination of signal molecules. Therefore, a signal molecule is selected such that the intensity of the signal molecules can be detected reliably and reproducibly, and optionally enhanced. Signal molecules whose signal intensity can be reliably and reproducibly detected and that can be associated with probes have been disclosed (See e.g., Vo-Dinh et al, J. Raman Spectrosc,. 30: 785-793 (1999); Graham et al, Anal. Chem. 74:1069-1074 (2002), Mirkin et al, Science 297: 1536-1 540 (2002)). For example, a probe with one Rhodamine 6G (R6G) molecule can be distinguished from a probe with two R6G molecules.

Optionally, in order to calibrate the intensity from attached signal molecules, a signal molecule can be attached to every probe as an intensity reference signal molecule. In certain aspects, the reference signal molecule is identical in every probe of the population of probes. The reference signal molecule can be different than any of the encoding signal molecules, also referred to herein in certain aspects as encoding dyes, which are the detectably distinguishable molecules whose number and type identify the probe. Optical signals from the detectably distinguishable signal molecules, can be normalized by using the signal from this reference signal molecule.

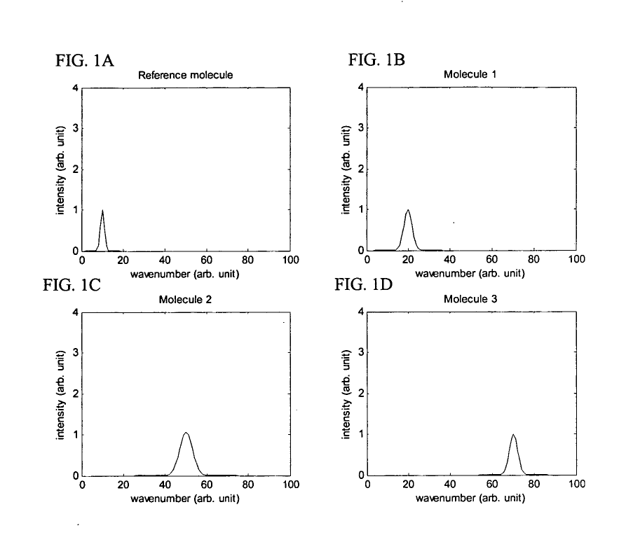

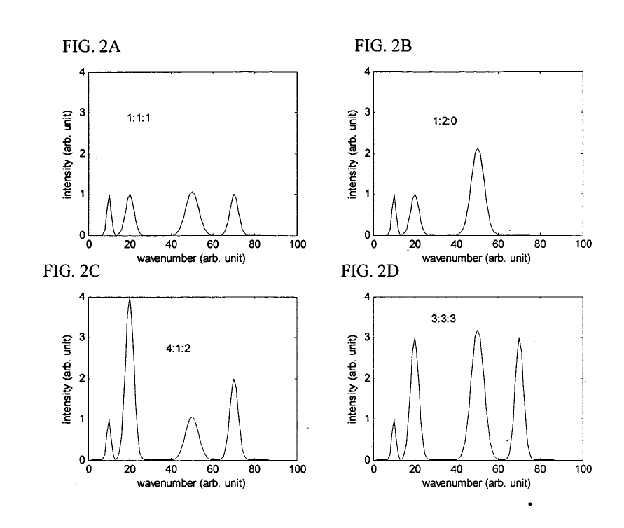

FIGS. 1A-D and 2A-D provide an illustrative example of the use of a reference molecule (FIG. 1A) to determine the copy number of 3 encoding signal molecules (FIGS. 1B-D). Each molecule has a unique peak (FIGS. 1A-D). By calibrating the intensity of the encoding molecules with the intensity of the reference molecule, the number of encoding molecules can be determined. For example, FIG. 2A illustrates a 1:1:1 ratio of signal molecules 1-3. FIG. 2B illustrates a 1:2:0 ratio of signal molecules. FIG. 2C illustrates a 4:1:2 ratio. And FIG. 2D, illustrates a 3:3:3 ratio. As illustrated in the series of Figures, based on the relative intensities between encoding signal molecules, and/or between the encoding signal molecules and the reference molecule, the number of molecules of each encoding signal molecule can be determined.

Non-limiting examples of reference signal molecules are listed in Table 1. Reference signal molecules assist in a determination of the number of each type of signal molecule present in a detected signal because a ratio of the signal intensity for the reference signal molecule to a known number of encoding signal molecules is known or can be determined.

TABLE 1
Exemplary reference signal molecules

| Organic Compound | Abbreviation |
|---|---|
| 2-Aminopurine | AP |
| 2-Fluoroadenine | FA |
| 4-Amino-pyrazolo[3,4-d]pyrimidine | APP |
| 4-Pyridinecarboxaldoxime | PCA |
| 8-Azaadenine | AA |
| Adenine | A |
| 4-Amino-3,5-di-2-pyridyl-4H-1,2,4-triazole | AMPT |
| 6-(g,g-Dimethylallylamino)purine | DAAP |
| Kinetin | KN |
| N6-Benzoyladenine | BA |
| Zeatin | ZT |
| 4-Amino-2,1,3-benzothiadiazole | ABT |
| Acriflavine | AF |
| Basic blue 3 | BB |
| Methylene Blue | MB |
| 2-Mercapto-benzimidazole | MBI |
| 4-Amino-6-mercaptopyrazolo[3,4- | AMPP |
| d]pyrimidine | |
| 6-Mercaptopurine | MP |
| 8-Mercaptoadenine (adenine thiol) | AT |
| 9-Aminoacridine | AN |
| Cyanine dyes | Cy3 |
| Ethidium bromide | Ebr |
| Fluorescein | FAM |
| Rhodamine Green | R110 |
| Rhodamine-6G | R6G |

In aspects where a reference signal molecule is not used, the number of probe molecules can be determined using another method. For example, the number of probe molecules can be determined using the absolute intensity of the signal molecules. The signal intensity from signal molecules increases proportionally with the number of signal molecules. If the instrument is calibrated with a known number of signal molecules, the number of signal molecules can be estimated from the absolute intensity of the signal molecules.

The present invention overcomes the problem in the art of attempting to simultaneously detect too many labels by using order-specific signal molecules. Each signal molecule is assigned to encode a subunit sequence, such as a target position of a template polynucleotide, rather than encoding each nucleotide using certain a unique dye.

By combining intensity signal detection with assigning a signal molecule to a target position, numerous combinations of signal molecules are generated that can be detected and differentiated optically. These combinations of signal molecules store information about the probes, such as oligonucleotide probes, to which they are associated. If m-types of signal molecules are used, and each type of signal molecule can be used up to j times in one series of detectably distinguishable signal molecules (i.e. tag), the number of possible variations are represented by j\hat{ }m. This covers all possible sequences in n-mer, 4\hat{ }n. (Thus, 4\hat{ }n=j\hat{ }m, or m=2n log 2/log j). The maximum number of signal molecules possibly used in one tag is j*m. Although the encoding can be done with the minimum number of signal molecules when j=3 (up to ˜5% reduction compared to when j=4), for simplicity we will describe the case when j=4 (each type of signal molecules can be used up to 4 times in one probe). When j=4, m equals n. For a 3-mer, 3 types of signal molecules are needed to represent all possible 3-mer sequences.

For sake of discussion, the following symbols are used to represent three types of signal molecules, \mathring{×}, ⊕, and \mathring{/}, \mathring{×} is used to encode the information of the first base in the 3-mer, ⊕ for the second base, and \mathring{/} for the third base. The optical signal from each type of signal molecule should be distinguishable (FIG. 2). Also, the information can be encoded in a way that the number of signal molecules of each kind represents the type of nucleotide. For example, one copy of a signal molecule can represent, A; two copies of the signal molecule can represent G; three copies for C; and four copies for T. Following this scheme all 64 possible sequences in 3-mer can be encoded (Table 2).

In this design, two types of linearity are assumed. First, for each type of signal molecule, the optical signal is proportional to the number of signal molecules of the very same kind. Second, the optical signal from one type of signal molecules does not alter the optical signal from other types of signal molecules. Numerous combinations of signal molecules are known that meet these properties. For example, all 25 molecules in Table 1 can be used as signal molecules, as each molecule has a unique Raman signature that increases proportionally to the number of molecules and is not altered by the presence of other signal molecules.

Thus, optical signal from the signal molecules can be considered as a linear superposition of optical signals from each individual signal molecule. Please note that the actual order of the signal molecules may not matter. \mathring{×} ⊕ \mathring{/} \mathring{/}, \mathring{/} \mathring{×} \mathring{/} ⊕, ⊕ \mathring{/} \mathring{×} \mathring{/}, and ⊕ \mathring{/} \mathring{/} \mathring{×} will all yield the same optical signal. Furthermore, these signal molecules do not have to be positioned in a specific arrangement for reading. As long as they are positioned inside the collection volume, all their signals will be collected.

For a 20-mer (i.e. a 20 subunit polymer such as an oligonucleotide 20 nucleotides in length) and j=4, 1 to 4 copies of 20 different signal molecules (i.e. 80 total combinations of identity and number of signal molecules) can be used to encode all the 20-mer sequences. Optionally, 1 signal molecule can be used as an intensity reference signal molecule. The 80 total combinations of 20 unique signal molecules is a great reduction from 10¹² types of signal molecules needed if the encoding method of the present invention was not used. Accordingly, in this aspect of the invention, each unique signal molecule is used up to 4 times per probe. Furthermore, the number of unique signal molecules is equal to the number of nucleotides of the probe. In addition, in this aspect, the nucleotide occurrence of each nucleotide position of a probe is identified by a number of copies of a unique signal molecule.

For the sequence recovery process, the optical signal from the tag can be decomposed to identify the intensity contribution from each type of signal molecule. If each signal molecule has multiple peaks, it may be difficult to identify a peak that uniquely originates from only one signal molecule. Multivariate least-squares analysis can decompose the spectrum of tags into its components and estimate the number of signal molecules (See e.g., R. Kramer, Chemometric Techniques for Quantitative Analysis (New York: Marcel Dekker, 1998)). Thus, peak intensity measurements and multivariate least-squares methods can be used for the decomposition process.

This information can be used to find the matching sequence from a look up table. Table 2 exemplifies a look-up table for a 3-mer.

TABLE 2
An exemplary nucleic acid sequence encoding table for a 3-mer

| | | | |
|---|---|---|---|
| AAA [symbol] ⊕ [symbol] | GAA [symbol] [symbol] ⊕ [symbol] | CAA [symbol] [symbol] [symbol] ⊕ [symbol] | TAA [symbol] [symbol] [symbol] [symbol] ⊕ [symbol] |
| AAG [symbol] ⊕ [symbol] [symbol] | GAG [symbol] [symbol] ⊕ [symbol] [symbol] | CAG [symbol] [symbol] [symbol] ⊕ [symbol] [symbol] | TAG [symbol] [symbol] [symbol] [symbol] ⊕ [symbol] [symbol] |
| AAC [symbol] ⊕ [symbol] [symbol] [symbol] | GAC [symbol] [symbol] ⊕ [symbol] [symbol] [symbol] | CAC [symbol] [symbol] [symbol] ⊕ [symbol] [symbol] [symbol] | TAC [symbol] [symbol] [symbol] [symbol] ⊕ [symbol] [symbol] [symbol] |
| AAT [symbol] ⊕ [symbol] [symbol] [symbol] [symbol] | GAT [symbol] [symbol] ⊕ [symbol] [symbol] [symbol] [symbol] | CAT [symbol] [symbol] [symbol] ⊕ [symbol] [symbol] [symbol] [symbol] | TAT [symbol] [symbol] [symbol] [symbol] ⊕ [symbol] [symbol] [symbol] [symbol] |
| AGA [symbol] ⊕ ⊕ [symbol] | GGA [symbol] [symbol] ⊕ ⊕ [symbol] | CGA [symbol] [symbol] [symbol] ⊕ ⊕ [symbol] | TGA [symbol] [symbol] [symbol] [symbol] ⊕ ⊕ [symbol] |
| AGG [symbol] ⊕ ⊕ [symbol] [symbol] | GGG [symbol] [symbol] ⊕ ⊕ [symbol] [symbol] | CGG [symbol] [symbol] [symbol] ⊕ ⊕ [symbol] [symbol] | TGG [symbol] [symbol] [symbol] [symbol] ⊕ ⊕ [symbol] [symbol] |
| AGC [symbol] ⊕ ⊕ [symbol] [symbol] [symbol] | GGC [symbol] [symbol] ⊕ ⊕ [symbol] [symbol] [symbol] | CGC [symbol] [symbol] [symbol] ⊕ ⊕ [symbol] [symbol] [symbol] | TGC [symbol] [symbol] [symbol] [symbol] ⊕ ⊕ [symbol] [symbol] [symbol] |
| AGT [symbol] ⊕ ⊕ [symbol] [symbol] [symbol] [symbol] | GGT [symbol] [symbol] ⊕ ⊕ [symbol] [symbol] [symbol] [symbol] | CGT [symbol] [symbol] [symbol] ⊕ ⊕ [symbol] [symbol] [symbol] [symbol] | TGT [symbol] [symbol] [symbol] [symbol] ⊕ ⊕ [symbol] [symbol] [symbol] [symbol] |
| ACA [symbol] ⊕ ⊕ ⊕ [symbol] | GCA [symbol] [symbol] ⊕ ⊕ ⊕ [symbol] | CCA [symbol] [symbol] [symbol] ⊕ ⊕ ⊕ [symbol] | TCA [symbol] [symbol] [symbol] [symbol] ⊕ ⊕ ⊕ [symbol] |
| ACG [symbol] ⊕ ⊕ ⊕ [symbol] [symbol] | GCG [symbol] [symbol] ⊕ ⊕ ⊕ [symbol] [symbol] | CCG [symbol] [symbol] [symbol] ⊕ ⊕ ⊕ [symbol] [symbol] | TCG [symbol] [symbol] [symbol] [symbol] ⊕ ⊕ ⊕ [symbol] [symbol] |
| ACC [symbol] ⊕ ⊕ ⊕ [symbol] [symbol] [symbol] | GCC [symbol] [symbol] ⊕ ⊕ ⊕ [symbol] [symbol] [symbol] | CCC [symbol] [symbol] [symbol] ⊕ ⊕ ⊕ [symbol] [symbol] [symbol] | TCC [symbol] [symbol] [symbol] [symbol] ⊕ ⊕ ⊕ [symbol] [symbol] [symbol] |
| ACT [symbol] ⊕ ⊕ ⊕ [symbol] [symbol] [symbol] [symbol] | GCT [symbol] [symbol] ⊕ ⊕ ⊕ [symbol] [symbol] [symbol] [symbol] | CCT [symbol] [symbol] [symbol] ⊕ ⊕ ⊕ [symbol] [symbol] [symbol] [symbol] | TCT |
| | | | [symbol] [symbol] [symbol] [symbol] ⊕ ⊕ ⊕ [symbol] [symbol] [symbol] [symbol] |
| ATA [symbol] ⊕ ⊕ ⊕ ⊕ [symbol] | GTA [symbol] [symbol] ⊕ ⊕ ⊕ ⊕ [symbol] | CTA [symbol] [symbol] [symbol] ⊕ ⊕ ⊕ ⊕ [symbol] | TTA [symbol] [symbol] [symbol] [symbol] ⊕ ⊕ ⊕ ⊕ [symbol] |
| ATG [symbol] ⊕ ⊕ ⊕ ⊕ [symbol] [symbol] | GTG [symbol] [symbol] ⊕ ⊕ ⊕ ⊕ [symbol] [symbol] | CTG [symbol] [symbol] [symbol] ⊕ ⊕ ⊕ ⊕ [symbol] [symbol] | TTG [symbol] [symbol] [symbol] [symbol] ⊕ ⊕ ⊕ ⊕ [symbol] [symbol] |
| ATC [symbol] ⊕ ⊕ ⊕ ⊕ [symbol] [symbol] [symbol] | GTC [symbol] [symbol] ⊕ ⊕ ⊕ ⊕ [symbol] [symbol] [symbol] | CTC [symbol] [symbol] [symbol] ⊕ ⊕ ⊕ ⊕ [symbol] [symbol] [symbol] | TTC |
| | | | [symbol] [symbol] [symbol] [symbol] ⊕ ⊕ ⊕ ⊕ [symbol] [symbol] [symbol] |
| ATT | GTT | CTT | TTT |
| [symbol] ⊕ ⊕ ⊕ ⊕ [symbol] [symbol] [symbol] [symbol] | [symbol] [symbol] ⊕ ⊕ ⊕ ⊕ [symbol] [symbol] [symbol] [symbol] | [symbol] [symbol] [symbol] ⊕ ⊕ ⊕ ⊕ [symbol] [symbol] [symbol] [symbol] | [symbol] [symbol] [symbol] [symbol] ⊕ ⊕ ⊕ ⊕ [symbol] [symbol] [symbol] [symbol] |

For non-optical detection, the size, shape, and other detectable properties of particles, depending on the method of detection, as discussed further herein, can be varied to produce multiple types of nanotags, also referred to herein as nanoparticles. For example, the image of three signal molecules, ♦•• has the same sequence information as •♦•, •♦•, or even non-linear configurations. Accordingly, in certain aspects, the signal molecules are a series of nanotags. Furthermore, in certain aspects each nanotag in the series of nanotags is of detectably distinguishable size and/or shape. In the methods of the present invention the intensity of the signal obtained from each individual nanotag is determined and used to determine the number of copies of each nanotag, which identifies the probe.

In another embodiment, a method for identifying one or more target molecules is provided, wherein a target molecule is contacted with a population of labeled probes that each include a series of associated signal molecules whose copy number and type identify the probes. The number of probes exceeds the number of unique signal molecules and each unique signal molecule is detectably distinguishable. Probes that bind the target molecule are separated from unbound probes. The signal from the bound probe is detected and decomposed into the number and type of signal molecules in the bound probes, thereby identifying the target molecule.

The probe is a specific binding pair member that binds the target molecule, which is the other member of the specific binding pair that includes the probe. Furthermore, the target molecule in certain aspects of the invention, is a target polymer that includes a chain of subunits. In these embodiments, for example, the probe can bind specifically to certain subunits of the polymer. Thus, the method in certain aspects, identifies the presence of specific subunits of a polymer, for example the presence of a nucleotide sequence with a nucleic acid. The methods of this embodiment can be used for many different methods, for example methods used in biotechnology and/or health care including DNA sequencing, immunoassays, single nucleotide polymorphism (SNP) detection, specific genotype detection, and ligand binding.

In aspects of the present invention wherein the target molecule is a polymer, the polymer is, for example, a polypeptide, a polynucleotide, or a polysaccharide. For example, where the target molecule is a polypeptide, the specific bind pair member is an antibody. On the other hand, where the target molecule is a nucleic acid molecule, for example a single-stranded nucleic acid molecule, the specific bind pair member, (i.e. the probe) is typically an oligonucleotide that binds to the polynucleotide.

In certain aspects, the target molecule is a protein and the probe is, for example, an antibody. In another aspect, the probe is a ligand and the target molecule is, for example, a receptor. In another aspect, the target molecule is a polynucleotide and the probe is, for example, a polynucleotide that binds the polynucleotide.

The method can be used to detect one or more different target molecules. For example, the method can be used to detect 2 or more (i.e. a population of target molecules), 3 or more, 4 or more, 5 or more, 10 or more, 25 or more, 50 or more, 100 or more, 250 or more, 500 or more, or 1000 or more different target molecules.

The method can be used to identify a nucleotide occurrence at a target nucleotide position of a target nucleic acid, for example. In this aspect, the target nucleotide can be a site of a polymorphism such as a single nucleotide polymorphism. Furthermore, the nucleotide occurrence for multiple target nucleotide positions can be identified. For example, the nucleotide occurrence at 2, 3, 4, 5, 10, 20, 25, 50, 100, 250, 500, 1000, 2500, 5000, or 10000 positions can be determined. For these aspects, the population of labeled oligonucleotide probes can include nucleotide sequences that are complementary to every known or every possible nucleotide occurrence at the target nucleotide positions. This approach provides the possibility of determining the nucleotide occurrence at many SNPs in a single reaction.

Polymorphisms are allelic variants that occur in a population. A polymorphism can be a single nucleotide difference present at a locus, or can be an insertion or deletion of one or a few nucleotides. As such, a single nucleotide polymorphism (SNP) is characterized by the presence in a population of one or two, three or four nucleotide occurrences (i.e., adenosine, cytosine, guanosine or thymidine) at a particular locus in a genome such as the human genome. As indicated herein, methods of the invention in certain aspects, provide for the detection of a nucleotide occurrence at a SNP location or a detection of both genomic nucleotide occurrences at a SNP location for a diploid organism such as a mammal.

In certain aspects of this embodiment of the invention wherein the target molecule is a target nucleic acid, one or more, two or more, three of more, four or more, five or more, ten or more, twenty or more, twenty-five or more, fifty or more, one-hundred or more, two-hundred fifty or more, five hundred or more, one-thousand or more, target nucleic acid sequences are identified that are complementary to labeled oligonucleotides. In certain aspects of the invention, the population of probes includes a probe that binds to every possible subunit in the polymer. In another aspect, the probes are oligonucleotides of an identical length. For example, the population of probes can individually encode every possible sequence for the given length. These aspects of the invention can be used, for example, to determine nucleotide sequence information of a target polynucleotide.

In another embodiment, a method for detecting a nucleotide, nucleoside, or base is provided, wherein the nucleotide, nucleoside, or base are deposited on a substrate that includes metallic nanoparticles, a metal-coated nanostructure, or a substrate that includes aluminum, before irradiated the deposited nucleotide, nucleoside or base with a laser beam, and detecting the resulting Raman spectra. The detection method is useful, for example, in methods of sequencing nucleic acids disclosed herein.

In certain aspects of the invention, a target nucleic acid is cleaved into overlapping fragments and each of the overlapping fragments are sequenced using the methods provided herein. The sequences of individual fragments are aligned in order to determine the nucleotide sequence of the target nucleic acid. The target nucleic acid can be fragmented into fragments that are equal to or less than, for example, about 1000 nucleotides, 500 nucleotides, 250 nucleotides, 100 nucleotides, 50 nucleotides, or 25 nucleotides in length. In certain aspects, the fragments are less than twice the length of labeled oligonucleotide probes used to determine a nucleic acid sequence.

Accordingly, a method for detecting the occurrence of a target nucleotide sequence in a target nucleic acid is provided, wherein the target nucleic acid is contacted by two or more labeled probes that each include an oligonucleotide of a substantially identical or identical number of nucleotides associated with a series of detectably distinguishable signal molecules, wherein the nucleotide sequence of the oligonucleotide is identifiable by the number and type of detectably distinguishable signal molecules associated with the oligonucleotide, and wherein the number of probes in the population exceeds the number of unique signal molecules. Labeled probes that bind to the target nucleic acid are separated from unbound probes. A signal generated from the bound labeled probes is detected, thereby detecting the occurrence of the target nucleotide sequence in the polynucleotide.

The detected signal is decomposed to identify the number and type of signal molecules in the bound probes. The population of probes for this embodiment of the invention are discussed above. For example, in certain aspects, five or more oligonucleotide probes are provided. In another aspect, the population of probes includes all of the possible nucleotide sequence combinations for an oligonucleotide probe of a given length.

In another embodiment, the present invention provides a reaction mixture for a polynucleotide hybridization reaction that includes a target polynucleotide and a population of labeled oligonucleotide probes, wherein each labeled oligonucleotide probe includes an oligonucleotide associated with a series of detectably distinguishable signal molecules, wherein the nucleotide sequence of each oligonucleotide is represented by the number and type of detectably distinguishable signal molecules associated with the oligonucleotide, wherein the number of probes exceeds the number of unique signal molecules, and wherein each signal molecule is detectably distinguishable.

As discussed above, the population of labeled oligonucleotide probes includes, for example, at least 2, 3, 4, 5, 6, 7, 8, 9, 10, 15, 20, 25, 50, 75, 100 labeled probes. In certain embodiments, the population of labeled probes includes all of the possible sequence combinations for a population of probes of a given length. These aspects of the invention that includes all possible sequence combinations, are useful for example in sequencing by hybridization reactions.

The population of labeled oligonucleotide probes typically includes probes of the same length. For example, the population of labeled probes includes probes of an identical length of between 2 and 50 nucleotides, or for example an identical length of between about 3 and 25 nucleotides in length. For example, the population of labeled oligonucleotide probes can include all possible oligonucleotide probes 3 nucleotides in length. It will be recognized that although data analysis may be more complicated, the population of labeled oligonucleotide probes can have different lengths.

In another embodiment, a method for determining the nucleotide sequence of a target nucleic acid is provided, wherein the target nucleic acid is contacted with a population of labeled oligonucleotide probes, each labeled oligonucleotide probe including an oligonucleotide of an identical number of nucleotides associated with a series of detectably distinguishable signal molecules, wherein the nucleotide sequence of the oligonucleotide is identifiable by the number and type of signal molecules associated with the oligonucleotide. The number of probes typically exceeds the number of unique signal molecules, wherein the nucleotide sequence of the population of probes includes all of the possible nucleotide sequence combinations. A method according to this embodiment is a sequencing by hybridization reaction. The target polynucleotide is contacted with the population of labeled oligonucleotide probes to allow labeled oligonucleotide probes to bind to complementary sequences on the target polynucleotide. A signal generated from the bound probes is detected. The signal is decomposed to identify the number and type of signal molecules in the bound probes, thereby identifying the nucleotide sequence of the bound probes. The identity of the bound probes is then used to determine the nucleotide sequence of at least a portion of target polynucleotide using known methods for sequencing by hybridization reactions.

As discussed above, the signal molecules can be identified by either optical or non-optical methods. For example, the signal molecules can be detected using Raman spectroscopy, for example surface enhanced Raman spectroscopy. Alternatively, the labeled oligonucleotide probes can be detected using scanning probe microscopy or electron microscopy. Furthermore, the labeled oligonucleotide probes can include an intensity reference signal molecules.

In certain aspects of the invention, a target molecule is isolated from a biological sample before it is detected by the methods of the present invention. The biological sample is, for example, urine, blood, plasma, serum, saliva, semen, stool, sputum, cerebral spinal fluid, tears, mucus, and the like.

In certain aspects, the biological sample is from a mammalian subject, for example a human subject. The biological sample can be virtually any biological sample, particularly a sample that contains RNA or DNA from a subject. The biological sample can be a tissue sample which contains, for example, 1 to 10,000,000; 1000 to 10,000,000; or 1,000,000 to 10,000,000 somatic cells. The sample need not contain intact cells, as long as it contains sufficient RNA or DNA for the methods of the present invention, which in some aspects require only 1 molecule of RNA or DNA. According to aspects of the present invention wherein the biological sample is from a mammalian subject, the biological or tissue sample can be from any tissue. For example, the tissue can be obtained by surgery, biopsy, swab, stool, or other collection method.

In other aspects, the biological sample contains a pathogen, for example a virus or a bacterial pathogen. In certain aspects, the target nucleic acid is purified from the biological sample before it is contacted with a probe, however. The isolated target nucleic acid can be contacted with a reaction mixture without being amplified.

Since methods of the present invention can utilize nanoscale signal molecules, referred to herein as nanotags, such as nanoparticles, and can utilize single molecule detection methods such as SERS and scanning probe detection methods, methods of the present invention in certain aspects, provide the advantage that a smaller number of copies of a labeled oligonucleotide can be detected than with traditional labeling methods. For example, 100 copies or less, 50 copies or less, 25 copies or less, 10 copies or less, 5 copies or less, 4 copies or less, 3 copies or less, 2 copies or less, or a single copy of a labeled probe, such as a labeled oligonucleotide probe, can be detected using methods of the present invention.

As used herein, “about“means within ten percent of a value. For example, “about 100” would mean a value between 90 and 110.

“Nucleic acid” encompasses DNA, RNA (ribonucleic acid), single-stranded, double-stranded or triple stranded and any chemical modifications thereof. Virtually any modification of the nucleic acid is contemplated. A “nucleic acid” can be of almost any length, from oligonucleotides of 2 or more bases up to a full-length chromosomal DNA molecule. Nucleic acids include, but are not limited to, oligonucleotides and polynucleotides. A “polynucleotide” as used herein, is a nucleic acid that includes at least 25 nucleotides.

“Coded probe” refers to a probe molecule attached to one or more nanocodes. A probe molecule is any molecule that exhibits selective and/or specific binding to one or more target molecules. In various embodiments of the invention, each different probe molecule can be attached to a specific number and type of detectably distinguishable signal molecule, so that binding of a particular probe can be identified.

In certain aspects of the invention, coded probes, for example oligonucleotides, are covalently or non-covalently attached to one or more nanocodes. The number of nanocode copies and the identity of the nanocode in these aspects, identifies the sequence of the oligonucleotide and/or nucleic acid. These coded probes are sometimes referred to herein as “coded oligonucleotides,” “labeled oligonucleotides,” or “coded oligonucleotide probes.”

As indicated herein, certain embodiments of the invention are not limited as to the type of probe molecules that can be used. In these embodiments, any probe molecule known in the art, including but not limited to oligonucleotides, nucleic acids, antibodies, antibody fragments, binding proteins, receptor proteins, peptides, lectins, substrates, inhibitors, activators, ligands, hormones, cytokines, etc. can be used.

“Nanotags” are nanoscale molecules that can be detected using an optical or non-optical methods that are capable of detecting nanoscale molecules, such as SERS and scanning probe methods. “Nanocodes” include one or more submicrometer metallic barcodes, carbon nanotubes, fullerenes or any other nanoscale moiety that can be detected and identified by scanning probe microscopy. Nanocodes are not limited to single moieties and in certain embodiments of the invention a nanocode can include, for example, two or more fullerenes attached to each other. Where the moieties are fullerenes, they can, for example, consist of a series of large and small fullerenes attached together in a specific order. The order of differently sized fullerenes in a nanocode can be detected by scanning probe microscopy and used, for example, to identify the sequence of an attached oligonucleotide probe.

As used herein, the term “specific binding pair member” refers to a molecule that specifically binds or selectively hybridizes to another member of a specific binding pair. Specific binding pair member include, for example, an oligonucleotide and a nucleic acid to which the oligonucleotide selectively hybridizes, or a protein and an antibody that binds to the protein.

A “target” or “analyte” molecule is any molecule that can bind to a labeled probe, including but not limited to nucleic acids, proteins, lipids and polysaccharides. In some aspects of methods, binding of a labeled probe to a target molecule can be used to detect the presence of the target molecule in a sample.

In methods of the present invention related to determining a nucleotide sequence, a nucleic acid, such as a polynucleotide, to be at least partially sequenced, is contacted with a series of labeled oligonucleotides. Nucleic acid molecules to be detected, identified and/or sequenced can he prepared by any technique known in the art. In certain embodiments of the invention, the nucleic acids are naturally occurring DNA or RNA molecules. Virtually any naturally occurring nucleic acid can be detected, identified and/or sequenced by the disclosed methods including, without limit, chromosomal, mitochondrial and chloroplast DNA and ribosomal, transfer, heterogeneous nuclear and messenger RNA. In some embodiments, the nucleic acids to be analyzed can be present in crude homogenates or extracts of cells, tissues or organs. In other embodiments, the nucleic acids can be partially or fully purified before analysis. In alternative embodiments, the nucleic acid molecules to be analyzed can be prepared by chemical synthesis or by a wide variety of nucleic acid amplification, replication and/or synthetic methods known in the art.

Methods of the present invention analyze nucleic acids that in some aspects are isolated from a cell. Methods for purifying various forms of cellular nucleic acids are known. (See, e.g., Guide to Molecular Cloning Techniques, eds. Berger and Kimmel, Academic Press, New York, N.Y., 1987; Molecular Cloning: A Laboratory Manual, 2nd Ed., eds. Sambrook, Fritsch and Maniatis, Cold Spring Harbor Press, Cold Spring Harbor, N.Y., 1989). The methods disclosed in the cited references are exemplary only and any variation known in the art can be used. In cases where single stranded DNA (ssDNA) is to be analyzed, ssDNA can be prepared from double stranded DNA (dsDNA) by any known method. Such methods can involve heating dsDNA and allowing the strands to separate, or can alternatively involve preparation of ssDNA from dsDNA by known amplification or replication methods, such as cloning into M13. Any such known method can be used to prepare ssDNA or ssRNA.

Although certain embodiments of the invention concern analysis of naturally occurring nucleic acids, such as polynucleotides, virtually any type of nucleic acid could be used. For example, nucleic acids prepared by various amplification techniques, such as polymerase chain reaction (PCR™) amplification, could be analyzed. (See U.S. Pat. Nos. 4,683,195, 4,683,202 and 4,800,159.) Nucleic acids to be analyzed can alternatively be cloned in standard vectors, such as plasmids, cosmids, BACs (bacterial artificial chromosomes) or YACs (yeast artificial chromosomes). (See, e.g., Berger and Kimmel, 1987; Sambrook et al., 1989.) Nucleic acid inserts can be isolated from vector DNA, for example, by excision with appropriate restriction endonucleases, followed by agarose gel electrophoresis. Methods for isolation of nucleic acid inserts are known in the art. The disclosed methods are not limited as to the source of the nucleic acid to be analyzed and any type of nucleic acid, including prokaryotic, bacterial, viral, eukaryotic, mammalian and/or human can be analyzed within the scope of the claimed subject matter.

In various embodiments of the invention, multiple copies of a single nucleic acid can be analyzed by labeled oligonucleotide probe hybridization, as discussed below. Preparation of single nucleic acids and formation of multiple copies, for example by various amplification and/or replication methods, are known in the art. Alternatively, a single clone, such as a BAC, YAC, plasmid, virus, or other vector that contains a single nucleic acid insert can be isolated, grown up and the insert removed and purified for analysis. Methods for cloning and obtaining purified nucleic acid inserts are well known in the art.

It will be recognized that the scope of certain embodiments of the present invention is not limited to analysis of nucleic acids, but also concerns analysis of other types of biomolecules, including but not limited to proteins, lipids and polysaccharides. Methods for preparing and/or purifying various types of biomolecules are known in the art and any such method can be used.

In certain aspects, the population of labeled oligonucleotide probes are a series of oligonucleotides that can be used in a sequencing by hybridization reaction. In sequencing by hybridization one or more labeled oligonucleotide probes of known sequence are hybridized to a target nucleic acid sequence. Binding of the labeled oligonucleotide to the target indicates the presence of a complementary sequence in the target strand. Multiple labeled oligonucleotides can be hybridized simultaneously to the target molecule and detected simultaneously. In alternative embodiments, bound oligonucleotide probes can be identified attached to individual target molecules, or alternatively multiple copies of a specific target molecule can be allowed to bind simultaneously to overlapping sets of probe sequences. Individual molecules can be scanned, for example, using known molecular combing techniques coupled to a detection mode. (See, e.g., Bensimon et al., Phys. Rev. Lett. 74:4754-57, 1995; Michalet et al., Science 277:1518-23, 1997; U.S. Pat. Nos. 5,002,867, 5,840,862; 6,054,327; 6,225,055; 6,248,537; 6,265,153; 6,303,296 and 6,344,319.)

In various embodiments of the invention, hybridization of a target nucleic acid to a labeled oligonucleotide library can be performed under stringent conditions that only allow hybridization between fully complementary nucleic acid sequences. Low stringency hybridization is generally performed at 0.15 M to 0.9 M NaCl at a temperature range of 20° C. to 50° C. High stringency hybridization is generally performed at 0.02 M to 0. 1 5 M NaCl at a temperature range of 50° C. to 70° C. It is understood that the temperature and/or ionic strength of an appropriate stringency are determined in part by the length of an oligonucleotide probe, the base content of the target sequences, and the presence of formamide, tetramethylammonium chloride or other solvents in the hybridization mixture. The ranges mentioned above are exemplary and the appropriate stringency for a particular hybridization reaction is often determined empirically by comparison to positive and/or negative controls. The person of ordinary skill in the art is able to routinely adjust hybridization conditions to allow for only stringent hybridization between exactly complementary nucleic acid sequences to occur.

It is unlikely that a given target nucleic acid will hybridize to contiguous probe sequences that completely cover the target sequence. Rather, multiple copies of a target can be hybridized to pools of labeled oligonucleotides and partial sequence data collected from each. The partial sequences can be compiled into a complete target nucleic acid sequence using publicly available shotgun sequence compilation programs. Partial sequences can also be compiled from populations of a target molecule that are allowed to bind simultaneously to a library of barcode probes, for example in a solution phase.

In certain embodiments of the invention, labeled probes, such as labeled oligonucleotides, can be detected while still attached to a target molecule. Given the relatively weak strength of the binding interaction between short oligonucleotide probes and target nucleic acids, such methods can be more appropriate where, for example, labeled probes have been covalently attached to the target molecule using cross-linking reagents.

In various embodiments of the invention, oligonucleotide probes can be DNA, RNA, or any analog thereof, such as peptide nucleic acid (PNA), which can be used to identify a specific complementary sequence in a nucleic acid. In certain embodiments of the invention one or more oligonucleotide probe libraries can be prepared for hybridization to one or more nucleic acid molecules. For example, a set of labeled oligonucleotide probes containing all 4096 or about 2000 non-complementary 6-mers, or all 16,384 or about 8,000 non-complementary 7-mers can be used. If non-complementary subsets of oligonucleotide probes are to be used, a plurality of hybridizations and sequence analyses can be carried out and the results of the analyses merged into a single data set by computational methods. For example, if a library comprising only non-complementary 6-mers were used for hybridization and sequence analysis, a second hybridization and analysis using the same target nucleic acid molecule hybridized to those labeled probe sequences excluded from the first library can be performed.

In certain aspects of the invention, the labeled oligonucleotide probe libraries include a random nucleic acid sequence in the middle of the labeled oligonucleotide probe attached to constant nucleic acid sequences at one or both ends. For example, a subset of 12-mer labeled oligonucleotide probes can be used that consists of a complete set of random 8-mer sequences attached to constant 2-mers at each end. These labeled oligonucleotide probe libraries can be subdivided according to their constant portions and hybridized separately to a nucleic acid, followed by analysis using the combined data of each different labeled oligonucleotide probe library to determine the nucleic acid sequence. The skilled artisan will realize that the number of sublibraries required is a function of the number of constant bases that are attached to the random sequences. An alternative embodiment can use multiple hybridizations and analyses with a single labeled oligonucleotide probe library containing a specific constant portion attached to random oligonucleotide sequences. For any given site on a nucleic acid, it is possible that multiple labeled oligonucleotide probes of different, but overlapping sequence could bind to that site in a slightly offset manner. Thus, using multiple hybridizations and analyses with a single library, a complete sequence of the nucleic acid could be obtained by compiling the overlapping, offset labeled oligonucleotide probe sequences.

Oligonucleotides of a population of labeled oligonucleotide can be prepared by any known method, such as by synthesis on an Applied Biosystems 381A DNA synthesizer (Foster City, Calif.) or similar instruments. Alternatively, oligonucleotides can be purchased from a variety of vendors (e.g., Proligo, Boulder, Colo.; Midland Certified Reagents, Midland, Tex.). In embodiments where oligonucleotides are chemically synthesized, the signal molecules, such as a nanocode, quantum dots, or a Raman and/or fluorescent label, can be covalently attached to one or more of the nucleotide precursors used for synthesis. Alternatively, the signal molecules, can be attached after the oligonucleotide probe has been synthesized. In other alternatives, the nanocode(s) can be attached concurrently with oligonucleotide synthesis.

In certain aspects of the invention, labeled oligonucleotide probes include peptide nucleic acids (PNAs). PNAs are a polyamide type of DNA analog with monomeric units for adenine, guanine, thymine, and cytosine. PNAs are commercially available from companies such as PE Biosystems (Foster City, Calif.). Alternatively, PNA synthesis can be performed with 9-fluoroenylmethoxycarbonyl (Fmoc) monomer activation and coupling using O-(7-azabenzotriazol-1-yl)-1,1,3,3-tetramethyluronium hexafluorophosphate (HATU) in the presence of a tertiary amine, N,N-diisopropylethylamine (DIEA). PNAs can be purified by reverse phase high performance liquid chromatography (RP-HPLC) and verified by matrix assisted laser desorption ionization—time of flight (MALDI-TOF) mass spectrometry analysis.

In certain aspects of the present invention, after a target molecule is contacted with a population of labeled probes, labeled probes that bind to the target molecule are isolated. The separation can be carried out using physical, chemical, electrical, or any other methods known in the art, such as high performance liquid chromatography (HPLC), gel permeation chromatography, gel electrophoresis, ultrafiltration and/or hydroxylapatite chromatography.

In certain embodiments, probes of the invention are aptamers. Aptamers are oligonucleotides derived by an in vitro evolutionary process called SELEX (e.g. Brody and Gold, Molecular Biotechnology 74:5-13, 2000). The SELEX process involves repetitive cycles of exposing potential aptamers (nucleic acid ligands) to a target, allowing binding to occur, separating bound from free nucleic acid ligands, amplifying the bound ligands and repeating the binding process. After a number of cycles, aptamers exhibiting high affinity and specificity against virtually any type of biological target can be prepared. Because of their small size, relative stability and ease of preparation, aptamers can be well suited for use as probes. Since aptamers are comprised of oligonucleotides, they can easily be incorporated into nucleic acid type barcodes. Methods for production of aptamers are well known (e.g., U.S. Pat. Nos. 5,270,163; 5,567,588; 5,670,637; 5,696,249; 5,843,653). Alternatively, a variety of aptamers against specific targets can be obtained from commercial sources (e.g, Somalogic, Boulder, Colo.). Aptamers are relatively small molecules on the order of 7 to 50 kDa.

In certain embodiments, the probe is an antibody. Methods of production of antibodies are also well known in the art (e.g., Harlow and Lane, Antibodies: A Laboratory Manual, Cold Spring Harbor Laboratory, Cold Spring Harbor, N.Y., 1988.) Monoclonal antibodies suitable for use as probes can also be obtained from a number of commercial sources. Such commercial antibodies are available against a wide variety of targets. Antibody probes can be conjugated to signal molecules using standard chemistries, as discussed below.

In certain embodiments of the invention, a signal molecule can be incorporated into a precursor prior to the synthesis of a coded probe. For oligonucleotide-based coded probes, internal amino-modifications for covalent attachment at adenine (A) and guanine (G) positions are contemplated. Internal attachment can also be performed at a thymine (T) position using a commercially available phosphoramidite. In some embodiments library segments with a propylamine linker at the A and G positions can be used to attach signal molecules to coded probes. The introduction of an internal aminoalkyl tail allows post-synthetic attachment of the signal molecule. Linkers can be purchased from vendors such as Synthetic Genetics (San Diego, Calif.). In one embodiment of the invention, automatic coupling using the appropriate phosphoramidite derivative of the signal molecule is also contemplated. Such signal molecules can be coupled to the 5′-terminus during oligonucleotide synthesis.

In general, signal molecules will be covalently attached to the probe in such a manner as to minimize steric hindrance with the signal molecules, in order to facilitate coded probe binding to a target molecule, such as hybridization to a nucleic acid. Linkers can be used that provide a degree of flexibility to the coded probe. Homo-or hetero-bifunctional linkers are available from various commercial sources.

The point of attachment to an oligonucleotide base will vary with the base. While attachment at any position is possible, in certain embodiments attachment occurs at positions not involved in hydrogen bonding to the complementary base. Thus, for example, attachment can be to the 5 or 6 positions of pyrimidines such as uridine, cytosine and thymine. For purines such as adenine and guanine, the linkage is can be via the 8 position. The claimed methods and compositions are not limited to any particular type of probe molecule, such as oligonucleotides. Methods for attachment of signal molecules to other types of probes, such as peptide, protein and/or antibody probes, are known in the art.

In certain aspects, a series of detectably distinguishable signal molecules are attached to an oligonucleotide at one point, for example a 3′ terminus. In these aspects, the signal molecules are linked to each other.

The embodiments of the invention are not limiting as to the type of signal molecule that can be used. It is contemplated that any type of signal molecules known in the art can be used. As discussed in the next sections, non-limiting examples of nanoparticles include carbon nanotubes, fullerenes and submicrometer metallic barcodes, as discussed in more detail herein.

Signal molecules of the present invention include, but are not limited to, conducting, luminescent, fluorescent, chemiluminescent, bioluminescent and phosphorescent moieties, quantum dots, nanoparticles, metal nanoparticles, gold nanoparticles, silver nanoparticles, chromogens, antibodies, antibody fragments, genetically engineered antibodies, enzymes, substrates, cofactors, inhibitors, binding proteins, magnetic particles and spin label compounds. (U.S. Pat. Nos. 3,817,837; 3,850,752; 3,939,350; 3,996,345; 4,277,437; 4,275,149; and 4,366,241.) Furthermore, the signal molecules, in certain aspects, can be quantum dots (Qdot Corporation (Hayward, Calif.). In one aspect, the signal molecule itself includes an oligonucleotide or a polynucleotide.

According to certain embodiments of the invention, signal molecules of labeled probes are detected using a single molecule level surface analysis technique. Single molecule level surface analysis techniques, techniques which detect a single molecule or a small number of molecules, include, for example, Scanning Tunneling Microscopy (STM), scanning optical microscopy, scanning capacitance microscopy, atomic force microscopy (AFM), chemical force microscopy (CFM), lateral force microscopy (LFM), field emission scanning electron microscopy (FE-SEM), transmission electron microscopy (TEM), scanning TEM, Auger electron spectroscopy (AES), X-ray photoelectron spectroscopy (XPS), time-of-flight secondary ion mass spectrometry (TOF-SIMS), vibrational spectroscopy, Raman spectroscopy, especially SERS, or fluorescence spectroscopy.

Typically, the signal molecules are distinguishable based on a physical, chemical, optical, or electrical property, as discussed herein. In one aspect, the single molecule level surface analysis techniques is AFM and the signal molecules are distinguishable based on a topographic property or viscoelectric property. In another aspect the single molecule level surface analysis techniques is CFM or LFM and the signal molecules are distinguishable based on chemical force. In another aspect, the single molecule level surface analysis techniques is STM and the signal molecules are distinguishable based on a topographic property or an electrical property. In yet another aspect, the single molecule level surface analysis techniques is FE-SEM and the signal molecules are distinguishable based on a topographic property. In yet another aspect, the single molecule level surface analysis techniques is TEM and the signal molecules are distinguishable based on a topographic property. In yet another aspect, the single molecule level surface analysis techniques is AES and the signal molecules are distinguishable based on a topographic property. In yet another aspect, the single molecule level surface analysis techniques is XPS and the signal molecules are distinguishable based on chemical composition or chemical functionalization. In yet another aspect, the single molecule level surface analysis techniques is TOF-SIMS and the signal molecules are distinguishable based on chemical composition. In yet another aspect, the single molecule level surface analysis techniques is Raman spectroscopy and the signal molecules are distinguishable based on a chemical property. In still another aspect, the single molecule level surface analysis techniques is fluorescence spectroscopy and the signal molecules are distinguishable based on a fluorescent property.

Signal molecules used in the methods and compositions of the invention include, but are not limited to, any composition detectable by a single molecule level surface analysis method and/or a scanning probe microscopy. The detection methods include optical or non-optical (e.g., electrical, spectrophotometric, photochemical, biochemical, immunochemical, or chemical) techniques. Signal molecules include, but are not limited to, conducting, luminescent, fluorescent, chemiluminescent, bioluminescent and phosphorescent moieties, quantum dots, nanoparticles, metal nanoparticles, gold nanoparticles, silver nanoparticles, chromogens, antibodies, antibody fragments, genetically engineered antibodies, enzymes, substrates, cofactors, inhibitors, binding proteins, magnetic particles and spin label compounds (U.S. Pat. Nos. 3,817,837; 3,850,752; 3,939,350; 3,996,345; 4,277,437; 4,275,149; and 4,366,241). For example, in one aspect, the signal molecules are a series of quantum dots, for example 4 different quantum dots (Qdot Corporation). In other aspects, the signal molecules are other than quantum dots.

In aspects where the detection technique is Raman spectroscopy, especially SERS, non-limiting examples of Raman-active signal molecules that can be used include TRIT (tetramethyl rhodamine isothiol), NBD (7-nitrobenz-2-oxa-1,3-diazole), Texas Red dye, phthalic acid, terephthalic acid, isophthalic acid, cresyl fast violet, cresyl blue violet, brilliant cresyl blue, para-aminobenzoic acid, erythrosine, biotin, digoxigenin, 5-carboxy-4′,5′-dichloro-2′,7′-dimethoxy fluorescein, TET (6-carboxy-2′,4,7,7′-tetrachlorofluorescein), HEX (6-carboxy-2′,4,4′,5′,7,7′-hexachlorofluorescein), Joe (6-carboxy4′,5′-dichloro-2′,7′-dimethoxyfluorescein) 5-carboxy-2′,4′,5′,7′-tetrachlorofluorescein, 5-carboxyfluorescein, 5-carboxy rhodamine, Tamra (tetramethylrhodamine), 6-carboxyrhodamine, Rox (carboxy-X-rhodamine), R6G (Rhodamine 6G), phthalocyanines, azomethines, cyanines (e.g. Cy3, Cy3.5, Cy5), xanthines, succinylfluoresceins, N,N-diethyl4-(5′-azobenzotriazolyl)-phenylamine and aminoacridine. Furthermore, the Raman active signal molecules can include those that have been identified for use in gene probes (See e.g., Graham et al., Chem. Phys. Chem., 2001; Isola et al., Anal. Chem., 1998). In one aspect, the Raman active signal molecules include those disclosed in Kneipp et al., Chem Reviews (1999). These and other Raman signal molecules can be obtained from commercial sources (e.g., Molecular Probes, Eugene, Oreg.). Furthermore, Raman active signal molecules include composite organic-inorganic nanoparticles (See Su et al., U.S. Ser. No. ______, filed Dec. 29, 2003 entitled “Composite Organic-Inorganic Nanoparticles”).

Polycyclic aromatic compounds in general can function as Raman active signal molecules. Other signal molecules that can be of use include cyanide, thiol, chlorine, bromine, methyl, phosphorus and sulfur. In certain embodiments, carbon nanotubes can be of use as Raman signal molecules. The use of signal molecules in Raman spectroscopy is known (e.g., U.S. Pat. Nos. 5,306,403 and 6,174,677).

Raman active signal molecules can be attached directly to probes or can be attached via various linker compounds. Nucleotides that are covalently attached to Raman signal molecules are available from standard commercial sources (e.g., Roche Molecular Biochemicals, Indianapolis, Ind.; Promega Corp., Madison, Wis.; Ambion, Inc., Austin, Tex.; Amersham Pharmacia Biotech, Piscataway, N.J.). Raman active signal molecules that contain reactive groups designed to covalently react with other molecules, for example nucleotides or amino acids, are commercially available (e.g., Molecular Probes, Eugene, Oreg.)

In methods involving Raman active signal molecules, such as dyes, Raman active signal molecules either bound to a probe or separated from a probe, in certain embodiments, are deposited on a SERS substrate before being detected by SERS. Methods for depositing Raman signal molecules on substrates are known in the art. A detection unit can be designed to detect and/or quantify nucleotides by Raman spectroscopy. Various methods for detection of nucleotides by Raman spectroscopy are known in the art. (See, e.g., U.S. Pat. Nos. 5,306,403; 6,002,471; 6,174,677). However, Raman detection of labeled or unlabeled nucleotides at the single molecule level has not previously been demonstrated. Variations on surface enhanced Raman spectroscopy (SERS) or surface enhanced resonance Raman spectroscopy (SERRS) have been disclosed. In SERS and SERRS, the sensitivity of the Raman detection is enhanced by a factor of 106 or more for molecules adsorbed on roughened metal surfaces, such as silver, gold, platinum, copper or aluminum surfaces.

Raman active labels used as the series of detectably distinguishable labels, in certain aspects include composite organic-inorganic nanoparticles (See Su et al., U.S. Ser. No. ______, filed Dec. 29, 2003, entitled “Composite Organic-Inorganic Nanoparticles” (referred to herein as COIN nanoparticles or “COINs”)). In certain aspects of sequencing by hybridization embodiments, either one or both the capture oligonucleotide probes and the labeled oligonucleotide probes are associated with COIN nanoparticles and detected using SERS.

COINs are Raman-active probe constructs that include a core and a surface, wherein the core includes a metallic colloid including a first metal and a Raman-active organic compound. The COINs can further comprise a second metal different from the first metal, wherein the second metal forms a layer overlying the surface of the nanoparticle. The COINs can further comprise an organic layer overlying the metal layer, which organic layer comprises the probe. Suitable probes for attachment to the surface of the SERS-active nanoparticles for this embodiment include, without limitation, antibodies, antigens, polynucleotides, oligonucleotides, receptors, ligands, and the like. However, for these embodiments, COINs are typically attached to an oligonucleotide probe.

The metal for achieving a suitable SERS signal is inherent in the COIN, and a wide variety of Raman-active organic compounds can be incorporated into the particle. Indeed, a large number of unique Raman signatures can be created by employing nanoparticles containing Raman-active organic compounds of different structures, mixtures, and ratios. Thus, the methods described herein employing COINs are useful for the simultaneous determination of nucleotide sequence information from more than one, and typically more than 10 target nucleic acids. In addition, since many COINs can be incorporated into a single nanoparticle, the SERS signal from a single COIN particle is strong relative to SERS signals obtained from Raman-active materials that do not contain the nanoparticles described herein. This situation results in increased sensitivity compared to Raman-techniques that do not utilize COINs.

COINs are readily prepared for use in the invention methods using standard metal colloid chemistry. The preparation of COINs also takes advantage of the ability of metals to adsorb organic compounds. Indeed, since Raman-active organic compounds are adsorbed onto the metal during formation of the metallic colloids, many Raman-active organic compounds can be incorporated into the COIN without requiring special attachment chemistry.

In general, the COINs used in the invention methods are prepared as follows. An aqueous solution is prepared containing suitable metal cations, a reducing agent, and at least one suitable Raman-active organic compound. The components of the solution are then subject to conditions that reduce the metallic cations to form neutral, colloidal metal particles. Since the formation of the metallic colloids occurs in the presence of a suitable Raman-active organic compound, the Raman-active organic compound is readily adsorbed onto the metal during colloid formation. This simple type of COIN is referred to as type I COIN. Type I COINs can typically be isolated by membrane filtration. In addition, COINs of different sizes can be enriched by centrifugation.

In alternative embodiments, the COINs can include a second metal different from the first metal, wherein the second metal forms a layer overlying the surface of the nanoparticle. To prepare this type of SERS-active nanoparticle, type I COINs are placed in an aqueous solution containing suitable second metal cations and a reducing agent. The components of the solution are then subject to conditions that reduce the second metallic cations so as to form a metallic layer overlying the surface of the nanoparticle. In certain embodiments, the second metal layer includes metals, such as, for example, silver, gold, platinum, aluminum, and the like. This type of COIN is referred to as type II COINs. Type II COINs can be isolated and or enriched in the same manner as type I COINs. Typically, type I and type II COINs are substantially spherical and range in size from about 20 nm to 60 nm. The size of the nanoparticle is selected to be very small with respect to the wavelength of light used to irradiate the COINs during detection.

Typically, organic compounds, such as oligonucleotides, are attached to a layer of a second metal in type II COINs by covalently attaching the organic compounds to the surface of the metal layer Covalent attachment of an organic layer to the metallic layer can be achieved in a variety ways well known to those skilled in the art, such as for example, through thiol-metal bonds. In alternative embodiments, the organic molecules attached to the metal layer can be crosslinked to form a molecular network.

The COIN(s) used in the invention methods can include cores containing magnetic materials, such as, for example, iron oxides, and the like. Magnetic COINs can be handled without centrifugation using commonly available magnetic particle handling systems. Indeed, magnetism can be used as a mechanism for separating biological targets attached to magnetic COIN particles tagged with particular biological probes.

In certain aspects, each oligonucleotide probe is labeled with a series of COIN particles that are linked to each other through polymer chains. The series of COIN particles in these aspects, is typically linked to the oligonucleotide at one position, such as the 3′ terminus. These aspects of the invention are expected to provide the advantage of creating less interference by the labels with oligonucleotide hybridization than aspects in which each label of the series is bound.

A non-limiting example of a detection unit is disclosed in U.S. Pat. No. 6,002,471. In this embodiment, the excitation beam is generated by either a frequency doubled Nd:YAG laser at 532 nm wavelength or a frequency doubled Ti:sapphire laser at 365 nm wavelength. Pulsed laser beams or continuous laser beams can be used. The excitation beam passes through confocal optics and a microscope objective, and is focused onto the reaction chamber. The Raman emission light from the nucleotides is collected by the microscope objective and the confocal optics and is coupled to a monochromator for spectral dissociation. The confocal optics includes a combination of dichroic filters, barrier filters, confocal pinholes, lenses, and mirrors for reducing the background signal. Standard full field optics can be used as well as confocal optics. The Raman emission signal is detected by a Raman detector. The detector includes an avalanche photodiode interfaced with a computer for counting and digitization of the signal. In certain embodiments, a mesh including silver, gold, platinum, copper or aluminum can be included in the reaction chamber or channel to provide an increased signal due to surface enhanced Raman or surface enhanced Raman resonance. Alternatively, nanoparticles that include a Raman-active metal can be included.

Alternative embodiments of detection units are disclosed, for example, in U.S. Pat. No. 5,306,403, including a Spex Model 1403 double-grating spectrophotometer equipped with a gallium-arsenide photomultiplier tube (RCA Model C31034 or Burle Industries Model C3103402) operated in the single-photon counting mode. The excitation source is a 514.5 nm line argon-ion laser from SpectraPhysics, Model 166, and a 647.1 nm line of a krypton-ion laser (Innova 70, Coherent).

Alternative excitation sources include a nitrogen laser (Laser Science Inc.) at 337 nm and a helium-cadmium laser (Liconox) at 325 nm (U.S. Pat. No. 6,174,677). The excitation beam can be spectrally purified with a bandpass filter (Corion) and can be focused on the reaction chamber using a 6X objective lens (Newport, Model L6X). The objective lens can be used to both excite the nucleotides and to collect the Raman signal, by using a holographic beam splitter (Kaiser Optical Systems, Inc., Model HB 647-26N1 8) to produce a right-angle geometry for the excitation beam and the emitted Raman signal. A holographic notch filter (Kaiser Optical Systems, Inc.) can be used to reduce Rayleigh scattered radiation. Alternative Raman detectors include an ISA HR-320 spectrograph equipped with a red-enhanced intensified charge-coupled device (RE-ICCD) detection system (Princeton Instruments). Other types of detectors can be used, such as charged injection devices, photodiode arrays or phototransistor arrays.

Any suitable form or configuration of Raman spectroscopy or related techniques known in the art can be used for detection of nucleotides, including but not limited to normal Raman scattering, resonance Raman scattering, surface enhanced Raman scattering, surface enhanced resonance Raman scattering, coherent anti-Stokes Raman spectroscopy (CARS), stimulated Raman scattering, inverse Raman spectroscopy, stimulated gain Raman spectroscopy, hyper-Raman scattering, molecular optical laser examiner (MOLE) or Raman microprobe or Raman microscopy or confocal Raman microspectrometry, three-dimensional or scanning Raman, Raman saturation spectroscopy, time resolved resonance Raman, Raman decoupling spectroscopy or UV-Raman microscopy.

Fluorescent signal molecules can be used as signal molecules. These fluorescent molecules include, but are not limited to, fluorescein, 5-carboxyfluorescein (FAM), 2′7′-dimethoxy-4′5′-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′-dimethylaminophenylazo) benzoic acid (DABCYL), and 5-(2′-aminoethyl)aminonaphthalene-1-sulfonic acid (EDANS). Other potential fluorescent signal molecules are known in the art (e.g., U.S. Pat. No. 5,866,336). A wide variety of fluorescent signal molecules can be obtained from commercial sources, such as Molecular Probes (Eugene, Oreg.). Methods of fluorescent detection of molecules are also well known in the art and any such known method can be used.

Luminescent signal molecules that can be used in barcodes associated with physical objects include, but are not limited to, rare earth metal cryptates, europium trisbipyridine diamine, a europium cryptate or chelate, Tb tribipyridine, diamine, dicyanins, La Jolla blue dye, allopycocyanin, allococyanin B, phycocyanin C, phycocyanin R, thiamine, phycoerythrocyanin, phycoerythrin R, an up-converting or down-converting phosphor, luciferin, or acridinium esters.

Nanoparticles can be used as signal molecules. Although gold or silver nanoparticles are most commonly used as signal molecules, any type or composition of nanoparticle can be used as a signal molecule. In one aspect, the nanoparticles are incrementally grown nanotags (See U.S. patent application No. ______, entitled “Programmable Molecule Barcodes,” filed Sep. 24, 2003). Incrementally grown nanotags include a code section and a probe section. The probe section is used to induce hybridization to the target nucleic acid strand so that the tag binds specifically to the target sequence. The code section is configured so that the signal is easy to detect and unique to the sequence of the probe Incrementally grown nanotags can be generated by attaching a code element one nucleotide at a time, wherein each code element represents a nucleotide of a nucleic acid. In another aspect, incrementally grown nanotags can be generated using a variety of short oligonucleotides of known sequence attached to one or more tags. The oligonucleotide-tag molecules can be assembled into a barcode by hybridization to a template molecule. The template can include a container section for oligonucleotide-tag hybridization and a probe section for binding to a target molecule, such as a target nucleic acid.

The methods of the present invention utilize nanoparticles that can be virtually any length, but are typically 0.5 nm-1 μm in all dimensions, and in certain examples are 1 nm-500 nm in all dimensions. For example, the nanoparticle is typically between 1 nm and 500 nm in length. Furthermore, the nanoparticles are typically soluble in aqueous and organic phases (amphiphilic).

The nanoparticles to be used can be random aggregates of nanoparticles (colloidal nanoparticles). Alternatively, nanoparticles can be cross-linked to produce particular aggregates of nanoparticles, such as dimers, trimers, tetramers or other aggregates. Aggregates containing a selected number of nanoparticles (dimers, trimers, etc.) can be enriched or purified by known techniques, such as ultracentrifugation in sucrose solutions.

Modified nanoparticles suitable for attachment to probes are commercially available, such as the Nanogold® nanoparticles from Nanoprobes, Inc. (Yaphank, N.Y.). Nanogold® nanoparticles can be obtained with either single or multiple maleimide, amine or other groups attached per nanoparticle. Such modified nanoparticles can be attached to barcodes using a variety of known linker compounds.

Signal molecules can include submicrometer-sized metallic signal molecules (e.g., Nicewarner-Pena et al., Science 294:137-141, 2001). Nicewarner-Pena et al. (2001) disclose methods of preparing multimetal microrods encoded with submicrometer stripes, comprised of different types of metal. This system allows for the production of a very large number of distinguishable signal molecules—up to 4160 using two types of metal and as many as 8×10⁵ with three different types of metal. Such signal molecules can be attached to barcodes and detected. Methods of attaching metal particles, such as gold or silver, to oligonucleotides and other types of molecules are known in the art (e.g., U.S. Pat. No. 5,472,881).

Fullerenes can also be used as barcode signal molecules. Methods of producing fullerenes are known (e.g., U.S. Pat. No. 6,358,375). Fullerenes can be derivatized and attached to other molecules by methods similar to those disclosed herein for carbon nanotubes.

Other types of known signal molecules that can be attached to probes and detected are contemplated. Non-limiting examples of signal molecules of potential use include quantum dots (e.g., Schoenfeld, et al., Proc. 7th Int. Conf. on Modulated Semiconductor Structures, Madrid, pp. 605-608, 1995; Zhao, et al., 1 st Int. Conf. on Low Dimensional Structures and Devices, Singapore, pp. 467-471, 1995). Quantum dots and other types of signal molecules can also be obtained from commercial sources (e.g., Quantum Dot Corp., Hayward, Calif.).

Carbon nanotubes, such as single-walled carbon nanotubes (SWNTs), can also be used as signal molecules. Nanotubes can be detected in embodiments that employ a single molecule level surface analysis method, for example, by Raman spectroscopy (e.g., Freisignal et al., Phys. Rev. B 62: R2307-R2310, 2000). The characteristics of carbon nanotubes, such as electrical or optical properties, depend at least in part on the size of the nanotube. Carbon nanotubes can be made by a variety of techniques as discussed herein.

Nucleotides or bases, for example adenine, guanine, cytosine, or thymine can be used as signal molecule, typically for probes other than oligonucleotides and nucleic acids. For example, peptide based probes can be associated with nucleotides or purine or pyrimidines bases. Other types of purines or pyrimidines or analogs thereof, such as uracil, inosine, 2,6-diaminopurine, 5-fluoro-deoxycytosine, 7 deaza-deoxyadenine or 7-deaza-deoxyguanine can also be used as signal molecules. Other signal molecules include base analogs. A base is a nitrogen-containing ring structure without the sugar or the phosphate. Such signal molecules can be detected by optical techniques, such as Raman or fluorescence spectroscopy. Use of nucleotide or nucleotide analog signal molecules can not be appropriate where the target molecule to be detected is a nucleic acid or oligonucleotide, since the signal molecule portion of the barcode can potentially hybridize to a different target molecule than the probe portion.

Amino acids can also be used as signal molecules. Amino acids of potential use as signal molecules include but are not limited phenylalanine, tyrosine, tryptophan, histidine, arginine, cysteine, and methionine,

Bifunctional cross-linking reagents can be used for various purposes, such as attaching signal molecules to probes. The bifunctional cross-linking reagents can be divided according to the specificity of their functional groups, e.g., amino, guanidino, indole, or carboxyl specific groups. Of these, reagents directed to free amino groups are popular because of their commercial availability, ease of synthesis and the mild reaction conditions under which they can be applied (U.S. Pat. Nos. 5,603,872 and 5,401,511). Cross-linking reagents of potential use include glutaraldehyde (GAD), bifunctional oxirane (OXR), ethylene glycol diglycidyl ether (EGDE), and carbodiimides, such as 1-ethyl-3-(3-dimethylaminopropyl) carbodiimide (EDC).

In certain aspects of methods of the invention, scanning probe microscopy (SPM) is used to detect nanocodes. The SPM detection is performed either in a dry state or in a wet state. For example, dried barcodes can be read by AFM or STM. Wet nanoparticles (i.e., non-dried) can be identified by fluidic AFM or fluidic STM. That is, the detection can be performed by analyzing and processing scanned SPM images. The information read and decoded can be stored in a separate data storage system or transferred to computer systems for further data processing.

Examples of scanning probe microscopy techniques include scanning tunneling microscopy (STM), atomic force microscopy (AFM), scanning capacitance microscopy, and scanning optical microscopy, as well as are known in the art.

In certain aspects of the present invention that utilize non-optical detection methods, such as scanning probe microscopy methods, isolated labeled probes, or signal molecules stripped from the probes, are deposited on the surface of a scanning probe microscopy (SPM) substrate. That is, full probe molecules can be deposited on the surface, or probes that have hybridized can be isolated/separated, and the signal molecule stripped away for separate reading and decoding in the absence of the probe molecule. For example, a polynucleotide can be separated from the isolated labeled oligonucleotides before detection of an associated nanoparticle.

For example, nanoparticles are captured in a micro-scale (or smaller scale) analytical system in a dry or wet state for SPM analysis or for a single molecule level surface analysis. If necessary, an appropriate immobilization and dispersion technique can be used to improve the SPM analysis. For example, in SPM methods a substrate surface treatment such as thiol-gold, polylysine, silanization/AP-mica, as well as Mg2+ and/or Ni2+ (See e.g., Proc. Natl. Acad. Sci. USA 94:496-501 (1997); Biochemistry 36:461 (1997); Analytical Sci. 17:583 (2001); Biophysical Journal 77:568 (1999); and Chem. Rev. 96:1533 (1996)) can be used to uniformly disperse and immobilize a labeled polynucleotide. The appropriate dispersion allows for single molecule level analysis to be performed for reading and decoding information.

In various embodiments of the invention, nanoparticle labeled probes and/or target molecules bound to labeled probes can be attached to a surface and aligned for analysis. In some embodiments, labeled probes can be aligned on a surface and the incorporated nanoparticles detected as discussed herein. In alternative embodiments, nanoparticles can be detached from the probe molecules aligned on a surface and detected. In certain embodiments, the order of labeled probes bound to an individual target molecule can be retained and detected, for example, by scanning probe microscopy. In other embodiments, multiple copies of a target molecule can be present in a sample and the identity and/or sequence of the target molecule can be determined by assembling all of the sequences of labeled probes binding to the multiple copies into an overlapping target molecule sequence. Methods for assembling, for example, overlapping partial nucleic acid or protein sequences into a contiguous sequence are known in the art. In various embodiments, nanoparticles can be detected while they are attached to probe molecules, or can alternatively be detached from the probe molecules before detection.

Methods and apparatus for attachment to surfaces and alignment of molecules, such as nucleic acids, oligonucleotide probes and/or nanocodes are known in the art (See, e.g., Bensimon et al., Phys. Rev. Lett. 74:4754-57, 1995; Michalet et al., Science 277:1518-23, 1997; U.S. Pat. Nos. 5,840,862; 6,054,327; 6,225,055; 6,248,537; 6,265,153; 6,303,296 and 6,344,319; see also U.S. patent application Ser. No. 10/251,152, filed Sep. 20, 2002, entitled “Controlled Alignment of Nanocodes Encoding; Specific Information for Scanning Probe Microscopy (SPM)”). Nanocodes, coded probes and/or target molecules can be attached to a surface and aligned using physical forces inherent in an air-water meniscus or other types of interfaces. This technique is generally known as molecular combing.

Non-limiting examples of surfaces include glass, functionalized glass, ceramic, plastic, polystyrene, polypropylene, polyethylene, polycarbonate, PTFE (polytetrafluoroethylene), PVP (polyvinylpyrrolidone), germanium, silicon, quartz, gallium arsenide, gold, silver, nylon, nitrocellulose or any other material known in the art that is capable of having target molecules, nanocodes and/or coded probes attached to the surface. Attachment can be either by covalent or noncovalent interaction. Although in certain embodiments of the invention the surface is in the form of a glass slide or cover slip, the shape of the surface is not limiting and the surface can be in any shape. In some aspects of the invention, the surface is planar.

In aspects of the present invention involving SPM, after the labeled probes or stripped signal molecules are deposited, the nanoparticles that are deposited are identified using SPM. This is accomplished by scanning the surface using SPM. This allows information retrieval and decoding. The identity of an associated probe is then determined based on the identified deposited signal molecules, typically a nanotag for these embodiments. The data, often in a form of scanned images, are analyzed and processed through standard or customized/specialized image processing or digital signal processing techniques and software such as software provided by SPM manufacturers or any other image/signal processing software available. The information read (and decoded) can be stored in a separate data storage system or transferred to computer systems for further data processing.

Methods for using the identification of hybridizing oligonucleotides to decode sequence information is known in the art. For example, the cited references related to sequencing by hybridization included herein provide detailed methods for decoding polynucleotide sequence information based on a sequencing by hybridization result. Data collected from multiple nanoparticle readings are used to determine the polynucleotide sequence. Bioinformatics companies and government agencies provide necessary tools, services, and other associated tools for data processing to determine DNA sequences (e.g., Affymetrix (Santa Clara, Calif.)).

In various embodiments of the invention, the target molecules to be analyzed can be immobilized prior to, subsequent to, and/or during probe binding. For example, target molecule immobilization may be used to facilitate separation of bound coded probes from unbound coded probes. In certain embodiments, target molecule immobilization may also be used to separate bound labeled probes from the target molecules before labeled probe detection and/or identification.

Although the following discussion is directed towards immobilization of nucleic acids, the skilled artisan will realize that methods of immobilizing various types of biomolecules are known in the art and may be used in the claimed methods. Nucleic acid immobilization may be used, for example, to facilitate separation of target nucleic acids from labeled probes and from unhybridized (i.e. unbound) labeled probes, and/or to facilitate separation of bound from unbound labeled probes. In a non-limiting example, target nucleic acids may be immobilized and allowed to hybridize to labeled oligonucleotide probes. The substrate containing bound nucleic acids is extensively washed to remove unhybridized labeled oligonucleotide probes and labeled oligonucleotide probes hybridized to other labeled oligonucleotide probes. Following washing, the hybridized labeled oligonucleotide probes can be removed from the immobilized target nucleic acids by heating to about 90 to 95° C. for several minutes. The isolated labeled oligonucleotide probes can then be attached to a surface and detected, for example by SERS or an SPM method.

Immobilization of nucleic acids can be achieved by a variety of methods known in the art. In an exemplary embodiment of the invention, immobilization can be achieved by coating a substrate with streptavidin or avidin and the subsequent attachment of a biotinylated nucleic acid (Holmstrom et al., Anal. Biochem. 209:278-283, 1993). Immobilization can also occur by coating a silicon, glass or other substrate with poly-E-Lys (lysine), followed by covalent attachment of either amino- or sulfhydryl-modified nucleic acids using bifunctional crosslinking reagents (Running et al., BioTechniques 8:276-277, 1990; Newton et al., Nucleic Acids Res. 21:1155-62, 1993). Amine residues can be introduced onto a substrate through the use of aminosilane for cross-linking.

Immobilization can take place by direct covalent attachment of 5′-phosphorylated nucleic acids to chemically modified substrates (Rasmussen et al., Anal. Biochem. 198:138-142, 1991). The covalent bond between the nucleic acid and the substrate is formed by condensation with a water-soluble carbodiimide or other cross-linking reagent. This method facilitates a predominantly 5′-attachment of the nucleic acids via their 5′-phosphates. Exemplary modified substrates would include a glass slide or cover slip that has been treated in an acid bath, exposing SiOH groups on the glass (U.S. Pat. No. 5,840,862).

DNA is commonly bound to glass by first silanizing the glass substrate, then activating with carbodiimide or glutaraldehyde. Alternative procedures can use reagents such as 3-glycidoxypropyltrimethoxysilane (GOP), vinyl silane or aminopropyltrimethoxysilane (APTS) with DNA linked via amino linkers incorporated either at the 3′ or 5′ end of the molecule. DNA can be bound directly to membrane substrates using ultraviolet radiation. Other non-limiting examples of immobilization techniques for nucleic acids are disclosed in U.S. Pat. Nos. 5,610,287, 5,776,674 and 6,225,068. Commercially available substrates for nucleic acid binding are available, such as Covalink, Costar, Estapor, Bangs and Dynal. The skilled artisan will realize that the disclosed methods are not limited to immobilization of nucleic acids and are also of potential use, for example, to attach one or both ends of oligonucleotide coded probes to a substrate.

The type of substrate to be used for immobilization of the nucleic acid or other target molecule is not limiting. In various embodiments of the invention, the immobilization substrate can be magnetic beads, non-magnetic beads, a planar substrate or any other conformation of solid substrate comprising almost any material. Non-limiting examples of substrates that can be used include glass, silica, silicate, PDMS (poly dimethyl siloxane), silver or other metal coated substrates, nitrocellulose, nylon, activated quartz, activated glass, polyvinylidene difluoride (PVDF), polystyrene, polyacrylamide, other polymers such as poly(vinyl chloride) or poly(methyl methacrylate), and photopolymers which contain photoreactive species such as nitrenes, carbenes and ketyl radicals capable of forming covalent links with nucleic acid molecules (See U.S. Pat. Nos. 5,405,766 and 5,986,076).

Bifunctional cross-linking reagents can be of use in various embodiments of the invention. The bifunctional cross-linking reagents can be divided according to the specificity of their functional groups, e.g., amino, guanidino, indole, or carboxyl specific groups. Of these, reagents directed to free amino groups are popular because of their commercial availability, ease of synthesis and the mild reaction conditions under which they can be applied. Exemplary methods for cross-linking molecules are disclosed in U.S. Pat. Nos. 5,603,872 and 5,401,511. Cross-linking reagents include glutaraldehyde (GAD), bifunctional oxirane (OXR), ethylene glycol diglycidyl ether (EGDE), and carbodiimides, such as 1-ethyl-3-(3-dimethylaminopropyl) carbodiimide (EDC).

As indicated herein, in certain aspects of the methods of the present invention, nanocodes are detected using scanning probe microscopes (SPM). Scanning probe microscopes (SPM) are a family of instruments that are used to measure the physical properties of objects on a micrometer and/or nanometer scale. Different modalities of SPM technology are available, discussed in more detail below. Any modality of SPM analysis can be used for coded probe detection and/or identification. In general, an SPM instrument uses a very small, pointed probe in very close proximity to a surface to measure the properties of objects. In some types of SPM instruments, the probe can be mounted on a cantilever that can be a few hundred microns in length and between about 0.5 and 5.0 microns thick. Typically, the probe tip is raster-scanned across a surface in an xy pattern to map localized variations in surface properties. SPM methods of use for imaging biomolecules and/or detecting molecules of use as signal molecules are known in the art (e.g., Wang et al., Amer. Chem. Soc. Lett., 12:1697-98. 1996; Kim et al., Appl. Surface Sci. 130, 230, 340 -132:602-609, 1998; Kobayashi et al., Appl. Surface Sci. 157:228-32, 2000; Hirahara et al., Phys. Rev. Lett. 85:5384-87 2000; Klein et al., Applied Phys. Lett. 78:2396-98, 2001; Huang et al, Science 291:630-33, 2001; Ando et al., Proc. Natl. Acad. Sci. USA 12468-72, 2001). SPM methods that can be used to detect signal molecules of the present invention include Scanning tunneling microscopy (STM), atomic force microscopy (AFM), lateral force microscopy (LFM), chemical force microscopy (CFM), magnetic force microscopy (MFM), high frequency MFM, magnetoresistive sensitivity mapping (MSM), electric force microscopy (EFM), scanning capacitance microscopy (SCM), scanning spreading resistance microscopy (SSRM), tunneling AFM and conductive AFM. In certain of these modalities, magnetic properties of a sample can be determined. The skilled artisan will realize that metal signal molecules and other types of signal molecules can be designed that are identifiable by their magnetic as well as by electrical properties.

SPM instruments of use for coded probe detection and/or identification are commercially available (e.g. Veeco Instruments, Inc., Plainview, N.Y.; Digital Instruments, Oakland, Calif.). Alternatively, custom designed SPM instruments can be used.

In certain embodiments of the invention, a system for detecting labeled probes can include an information processing and control system. The embodiments are not limiting for the type of information processing system used. Such a system can be used to analyze data obtained from an SPM instrument and/or to control the movement of the SPM probe tip, the modality of SPM imaging used and the precise technique by which SPM data is obtained. An exemplary information processing system can incorporate a computer comprising a bus for communicating information and a processor for processing information. In one embodiment, the processor is selected from the Pentium® family of processors, including without limitation the Pentium®II family, the Pentium® III family and the Pentium® 4 family of processors available from Intel Corp. (Santa Clara, Calif.). In alternative embodiments of the invention, the processor can be a Celeron®, an Itanium®, an X-Scale® or a Pentium Xeon® processor (Intel Corp., Santa Clara, Calif.). In various other embodiments of the invention, the processor can be based on Intel® architecture, such as Intel® IA-32 or Intel® IA-64 architecture. Alternatively, other processors can be used.

The computer can further comprise a random access memory (RAM) or other dynamic storage device, a read only memory (ROM) or other static storage and a data storage device such as a magnetic disk or optical disc and its corresponding drive. The information processing system can also comprise other peripheral devices known in the art, such a display device (e.g., cathode ray tube or Liquid Crystal Display), an alphanumeric input device (e.g., keyboard), a cursor control device (e.g., mouse, trackball, or cursor direction keys) and a communication device (e.g., modem, network interface card, or interface device used for coupling to Ethernet, token ring, or other types of networks).

In particular embodiments of the invention, an SPM (scanning probe microscopy) unit can be connected to the information processing system. Data from the SPM can be processed by the processor and data stored in the main memory. The processor can analyze the data from the SPM to identify and/or determine the sequences of coded probes attached to a surface. By overlapping sequences of overlapping labeled probes, the computer can compile a sequence of a target nucleic acid. Alternatively, the computer can identify different known biomolecule species present in a sample, based on the identities of coded probes attached to the surface.

In certain embodiments of the invention, custom designed software packages can be used to analyze the data obtained from a detection technique. In alternative embodiments of the invention, data analysis can be performed using an information processing system and publicly available software packages. Non-limiting examples of available software for DNA sequence analysis include the PRISM™ DNA Sequencing Analysis Software (Applied Biosystems, Foster City, Calif.), the Sequencher™ package (Gene Codes, Ann Arbor, Mich.), and a variety of software packages available through the National Biotechnology Information Facility on the worldwide web at nbif.org/links/l.4.1.php.

Apparatus for labeled probe preparation, use and/or detection can be incorporated into a larger apparatus and/or system. In certain embodiments, the apparatus can include a micro-electro-mechanical system (MEMS). MEMS are integrated systems including mechanical elements, sensors, actuators, and electronics. All of those components can be manufactured by microfabrication techniques on a common chip, of a silicon-based or equivalent substrate (e.g., Voldman et al., Ann. Rev. Biomed. Eng. 1:401-425, 1999). The sensor components of MEMS can be used to measure mechanical, thermal, biological, chemical, optical and/or magnetic phenomena to detect barcodes. The electronics can process the information from the sensors and control actuator components such pumps, valves, heaters, etc. thereby controlling the function of the MEMS.

The electronic components of MEMS can be fabricated using integrated circuit (IC) processes (e.g., CMOS or Bipolar processes). They can be patterned using photolithographic and etching methods for computer chip manufacture. The micromechanical components can be fabricated using compatible “micromachining” processes that selectively etch away parts of the silicon wafer or add new structural layers to form the mechanical and/or electromechanical components.

Basic techniques in MEMS manufacture include depositing thin films of material on a substrate, applying a patterned mask on top of the films by some lithographic methods, and selectively etching the films. A thin film can be in the range of a few nanometers to 100 micrometers. Deposition techniques of use can include chemical procedures such as chemical vapor deposition (CVD), electrodeposition, epitaxy and thermal oxidation and physical procedures like physical vapor deposition (PVD) and casting. Methods for manufacture of nanoelectromechanical systems can also be used (See, e.g., Craighead, Science 290:1532-36, 2000.)

In some embodiments, apparatus and/or detectors can be connected to various fluid filled compartments, for example microfluidic channels or nanochannels. These and other components of the apparatus can be formed as a single unit, for example in the form of a chip (e.g. semiconductor chips) and/or microcapillary or microfluidic chips. Alternatively, individual components can be separately fabricated and attached together. Any materials known for use in such chips can be used in the disclosed apparatus, for example silicon, silicon dioxide, polydimethyl siloxane (PDMS), polymethylmethacrylate (PMMA), plastic, glass, quartz, etc.

Techniques for batch fabrication of chips are well known in computer chip manufacture and/or microcapillary chip manufacture. Such chips can be manufactured by any method known in the art, such as by photolithography and etching, laser ablation, injection molding, casting, molecular beam epitaxy, dip-pen nanolithography, chemical vapor deposition (CVD) fabrication, electron beam or focused ion beam technology or imprinting techniques. Non-limiting examples include conventional molding, dry etching of silicon dioxide; and electron beam lithography. Methods for manufacture of nanoelectromechanical systems can be used for certain embodiments. (See, e.g., Craighead, Science 290:1532-36, 2000.) Various forms of microfabricated chips are commercially available from, e.g., Caliper Technologies Inc. (Mountain View, Calif.) and ACLARA BioSciences Inc. (Mountain View, Calif.).

In certain embodiments, part or all of the apparatus can be selected to be transparent to electromagnetic radiation at the excitation and emission frequencies used for barcode detection by, for example, Raman spectroscopy. Suitable components can be fabricated from materials such as glass, silicon, quartz or any other optically clear material. For fluid-filled compartments that can be exposed to various analytes, for example, nucleic acids, proteins and the like, the surfaces exposed to such molecules can be modified by coating, for example to transform a surface from a hydrophobic to a hydrophilic surface and/or to decrease adsorption of molecules to a surface. Surface modification of common chip materials such as glass, silicon, quartz and/or PDMS is known (e.g., U.S. Pat. No. 6,263,286). Such modifications can include, for example, coating with commercially available capillary coatings (Supelco, Bellafonte, Pa.), silanes with various functional (e.g. polyethyleneoxide or acrylamide, etc).

In certain embodiments, such MEMS apparatus can be use to prepare labeled probes, to separate formed labeled probes from unincorporated components, to expose labeled probes to targets, and/or to detect labeled probes bound to targets.

In another embodiment, the present invention provide kits that include a population of labeled oligonucleotide probes, wherein each labeled oligonucleotide probe includes a series of detectably distinguishable signal molecules associated with an oligonucleotide, wherein the oligonucleotide is identifiable by the number and type of associated signal molecules, and wherein the number of probes exceeds the number of unique signal molecules. In certain aspects, each unique signal molecule is present up to 4 times per labeled oligonucleotide probe. In these aspects, for example, the number of unique signal molecules is equal to the number of nucleotides of the labeled oligonucleotide probe. Furthermore, the nucleotide occurrence of each nucleotide position of the labeled oligonucleotide probe can be identified by a number of copies of each signal molecule, for example.

In certain aspects of the kits herein, each labeled oligonucleotide probe includes an intensity reference signal molecule. Furthermore, in certain aspects, the population of labeled oligonucleotide probes includes all possible sequence combinations of an oligonucleotide of the identical length.

The following examples are intended to illustrate but not limit the invention.

### EXAMPLE 1

### Use of Population of Labeled Oligonucleotide Probes to Identify a Target Nucleic Acid

This example illustrates making and using the encoding method and population of labeled oligonucleotide probes disclosed herein, to identify an 8 nucleotide target sequence in a target nucleic acid. It is well known in the field, that dye molecules containing N-hydroxysuccinimidyl ester group, such as 7-diethylaminocoumarin-3-carboxylic acid, succinimidyl ester (DEAC), Fluorescein-5-EX, succinimidyl ester (FITC), Cy3, Cy3.5, Cy5, Cy5.5, Cy7, Rhodamine Green (RG), 6-carboxytetramethylrhodamine, succinimidyl ester (6-TAMRA), 5-(and-6)-carboxyrhodamine 6G,succinimidyl ester (5(6)-CR6G), Texas Red(R)-X, succinimidyl ester (TxR), can be attached to an amine group of a nucleotide by known chemistry (Randolph and Waggoner, Nucleic Acid Research, 1997). A commonly used nucleotide for labeling is the reactive amine derivative of dUTP, 5-(3-Aminoallyl)-2′-deoxyuridine 5′-triphosphate, which can be easily incorporated into DNA by a polymerase enzyme, or can be attached to a spacer (commonly alkyl chain of 6 or more carbons).

In this example, DEAC is used to encode the base information for the first nucleotide, FITC for the second, Cy3 for the third, Cy3.5 for the fourth, Cy 5 for the fifth, Cy5.5 for the sixth, Cy7 for the seventh, and RG for the eighth nucleotide. The number of dye molecules indicates the type of nucleotide in each position. The presence of one dye molecule of each type indicates nucleotide adenosine (“A”); two dye molecules for guanosine (“G”), three dye molecules for cytidine (“C”), and four dye molecules for thymidine (“T”). For example, one DEAC molecule indicates that the first nucleotide is “A”. Two DEAC molecules indicate that the first nucleotide is “G”, three DEAC molecules indicate that the first nucleotide is “C”, and four DEAC molecules indicate that the first nucleotide is “T.”

In this example, the DNA probe with sequence “AAAAAAAA” is attached to a series of dye molecules, DEAC, FITC, Cy3, Cy3.5, Cy5, Cy5.5, Cy7, and RG. The number of each type of dye molecule is one. The dye molecules can be attached in a random order, via dUTP and spacer to the DNA sequence AAAAAAA. The DNA probe with sequence “TTTTTTTT” is attached to a series of dye molecules, DEAC, DEAC, DEAC, DEAC, FITC, FITC, FITC, FITC, Cy3, Cy3, Cy3, Cy3, Cy3.5, Cy3.5, Cy3.5, Cy3.5, Cy5, Cy5, Cy5, Cy5, Cy5.5, Cy5.5, Cy5.5, Cy5.5, Cy7, Cy7, Cy7, Cy7, RG, RG, RG, and RG. The DNA probe with sequence “AGCTAATG” is attached to a series of dye molecules, DEAC, FITC, FITC, Cy3, Cy3, Cy3, Cy3.5, Cy3.5, Cy3.5, Cy3.5, CyS, Cy5.5, Cy7, Cy7, Cy7, Cy7, RG, and RG. All possible combinations of 8-mer sequence can be encoded by 8 dye molecules. 65536 8-mer DNA probes are synthesized and attached to corresponding tags to encode the sequence information.

For analyzing the sequence of a target DNA, a spot on a substrate covered with immobilized capture probe of known DNA sequence is used. A capture probe has 8-mer single strand DNA sequence which can bind to the target DNA. Multiple copies of a target DNA digested into 16-mer are introduced to the substrate with capture probes. In this hypothetical example, the target DNA sequence is “5′AGAACTACTATGATCA3′” (SEQ ID NO:1). The target DNA can bind to 9 different capture probes: “3′TCTTGATG5′,” “3′CTTGATGA5′,” “3′TTGATGAT5′,” “3′TGATGATA5′,” “3′GATGATAC5′,” “3′ATGATACT5′,” “3′TGATACTA5′,” “3′GATACTAG5′,” and “3′ATACTAGT5′.”

To avoid binding of exact complementary probes within the population of labeled oligonucleotide probes to each other, the probes can be applied in two steps, with exact complements applied at different steps. Accordingly, the mixture of the first 32768 non-complementary labeled probes is introduced into the substrate with captured target DNA. Some of the labeled probe oligonucleotides will bind to the unbound capture probes. Some of the labeled probe oligonucleotides may bind to the single strand segment of the captured target DNA. The substrate is washed to remove unbound labeled probe oligonucleotides. The mixture of the remainder of the non-complementary labeled probes is introduced into the substrate. Again, some of the labeled probe oligonucleotides will bind to the unbound capture probes. Some of the labeled probe oligonucleotides may bind to the single strand segment of the captured target DNA. The substrate is washed to remove unbound labeled probe oligonucleotides. The labeled probe oligonucleotides bind to the target DNA captured at the above 9 spots. The labeled probe oligonucleotides of sequence “ATACTAGT” bind to the target DNA captured in the spot with the capture probe sequence of “TCTTGATG.” The labeled probe oligonucleotides with four different sequences, “TACTAGTA”, “TACTAGTG”, “TACTAGTC”, and “TACTAGTT” can bind to the target DNA captured in the spot with the capture probe sequence of “”CTTGATGA.” The target DNA bound to the capture probe “CTTGATGA” has 7-mer for the labeled probe oligonucleotides to bind, compared to the target DNA bound to the capture probe “TCTTGATG” which has 8-mer for the labeled probe oligonucleotides to bind. As the DNA binding force decreases for the shorter length of binding DNA, the amount of the labeled probe oligonucleotides that binds in the spot of the capture probe “CTTGATGA” is less than the amount that binds in the spot of the capture probe “TCTTGATG.” Similarly, the amount of the labeled probe oligonucleotides that bind to 6-mer, 5-mer, 4-mer, 3-mer, 2-mer, and 1-mer decreases in that order. Thus, the signal of the labeled probes bound to the other 8 capture probe spots are weaker than the signal of the labeled probe bound to the full 8-mer of the target DNA.

A ligase enzyme is introduced with buffer to ligate the labeled probe to the capture probe. The substrate is heated and washed to denature and remove unligated labeled probe oligonucleotides.

Raman spectrum of each spot is recorded by a Raman instrument. The capture probe “TCTTGATG” is ligated to the labeled probe oligonucleotides “ATACTAGT.” From the signal of the labeled probe, the sequence of the labeled probe “ATACTAGT” is known. From the location of the spot, the sequence of the capture probe “TCTTGATG” is known. Thus, we know that the target DNA should have a DNA sequence complementary to the sequence of the ligated probe, “3′TCTTGATGATACTAGT5′” (SEQ ID NO:2). The complementary sequence is “5′AGAACTACTATGATCA3′” (SEQ ID NO:1).

Although the invention has been described with reference to the above example, it will be understood that modifications and variations are encompassed within the spirit and scope of the invention. Accordingly, the invention is limited only by the following claims.

1. A population of labeled oligonucleotide probes, each labeled oligonucleotide probe comprising an oligonucleotide associated with a series of detectably distinguishable signal molecules, the number and type of signal molecules identifying the nucleotide sequence of the probe, the number of probes in the population exceeding the number of unique signal molecules.

2. The population of labeled oligonucleotide probes of claim 1, wherein each unique signal molecule is present up to 4 times per labeled oligonucleotide probe.

3. The population of labeled oligonucleotide probes of claim 2, wherein the number of unique signal molecules is equal to the number of nucleotides of the labeled oligonucleotide probe.

4. The population of labeled oligonucleotide probes of claim 3, wherein the nucleotide occurrence of each nucleotide position of a labeled oligonucleotide probe is identified by a number of copies of a unique signal molecule.

5. The population of labeled oligonucleotide probes of claim 1, wherein each labeled oligonucleotide probe comprises an intensity reference signal molecule.

6. The population of labeled oligonucleotide probes of claim 1, wherein each oligonucleotide is an identical length of about 10 to 50 nucleotides.

7. The population of labeled oligonucleotide probes of claim 1, wherein the signal molecules are Raman labels.

8. The population of labeled oligonucleotide probes of claim 7, wherein the series of signal molecules comprise a polymethine dye or a signal molecule of Table 1.

9. The population of labeled oligonucleotide probes of claim 1, wherein the signal molecules are fluorescent labels or quantum dots.

10. The population of labeled oligonucleotide probes of claim 1, wherein the signal molecules are a series of nanotags.

11. A method to identify a nucleotide sequence of a target nucleic acid, the method comprising:
a) contacting a target nucleic acid with a population of labeled oligonucleotide probes, each labeled oligonucleotide probe comprising a series of detectably distinguishable signal molecules associated with an oligonucleotide, the oligonucleotide being identifiable by the number and type of associated signal molecules, wherein the number of probes exceeds the number of unique signal molecules;
b) separating bound oligonucleotide probes from unbound labeled oligonucleotide probes;
c) detecting a signal generated from the bound labeled oligonucleotide probes; and
d) decomposing the signal to identify the number and type of signal molecules in the bound labeled oligonucleotide probes, thereby identifying a nucleotide sequence of the target nucleic acid.

12. The method of claim 11, wherein each unique signal molecule is present up to 4 times per labeled oligonucleotide probe.

13. The method of claim 12, wherein the number of unique signal molecules is equal to the number of nucleotides of the labeled oligonucleotide probe.

14. The method of claim 13, wherein the nucleotide occurrence of each nucleotide position of the labeled oligonucleotide probe is identified by a number of copies of a unique signal molecule.

15. The method of claim 11, wherein each labeled oligonucleotide probe comprises an intensity reference signal molecule.

16. The method of claim 11, wherein each oligonucleotide is an identical length of about 10 to 50 nucleotides.

17. The method of claim 11, wherein the population of labeled oligonucleotide probes comprises all possible sequence combinations of an oligonucleotide of the identical length.

18. The method of claim 11, wherein the signal molecules are Raman labels.

19. The method of claim 18, wherein the series of signal molecules comprise a polymethine dye or a signal molecule of Table 1.

20. The method of claim 11, wherein the signal molecules are fluorescent labels or quantum dots.

21. The method of claim 11, wherein the signal molecules are a series of nanotags.

22. The method of claim 11, further comprising contacting the target nucleic acid, or a fragment thereof, with a population of capture oligonucleotide probes bound to a substrate at a series of spot locations before contacting the target nucleic acid with the population of labeled oligonucleotide probes.

23. The method of claim 22, further comprising ligating labeled oligonucleotide probes with capture oligonucleotide probes that bind adjacent target segments of the target nucleic acid.

24. A reaction mixture, comprising a target polynucleotide and a population of labeled probes, wherein each labeled probe comprises an oligonucleotide associated with a series of detectably distinguishable signal molecules, the nucleotide sequence of each oligonucleotide being represented by the number and type of signal molecules associated with the oligonucleotide, wherein the number of probes exceeds the number of unique signal molecules.

25. The reaction mixture of claim 24, wherein each unique signal molecule is present up to 4 times per labeled oligonucleotide probe.

26. The reaction mixture of claim 25, wherein the number of unique signal molecules is equal to the number of nucleotides of the labeled oligonucleotide probe.

27. The reaction mixture of claim 26, wherein the nucleotide occurrence of each nucleotide position of the labeled oligonucleotide probe is identified by a number of copies of a unique signal molecule.

28. The reaction mixture of claim 24, wherein each labeled oligonucleotide probe comprises an intensity reference signal molecule.

29. The reaction mixture of claim 24, wherein each oligonucleotide is an identical length of about 10 to 50 nucleotides.

30. The reaction mixture of claim 24, wherein the population of labeled oligonucleotide probes comprises all possible sequence combinations of an oligonucleotide of the identical length.

31. The reaction mixture of claim 24, wherein the signal molecules are Raman labels.

32. The reaction mixture of claim 31, wherein the series of signal molecules comprise a polymethine dye or a signal molecule of Table 1.

33. The reaction mixture of claim 24, wherein the signal molecules are fluorescent labels.

34. The reaction mixture of claim 24, wherein the signal molecules are a series of nanotags.


SEQUENCE LISTING (2 sequences)

SEQ ID NO: 1 - DNA, 16 bp, Artificial sequence
  Target sequence
agaactactatgatca

SEQ ID NO: 2 - DNA, 16 bp, Artificial sequence
  Probe
tgatcatagtagttct